(19) United States
(12) Patent Application Publication
(10) Pub. No.: US 2014/0039143 A1
(43) Pub. Date: Feb. 6, 2014

(54) PROCESS FOR PRODUCING PROPYLENE FROM SYNGAS VIA FERMENTATIVE PROPANOL PRODUCTION AND DEHYDRATION

(71) Applicant: Walter Vermeiren, Houthalen (BE)

(72) Inventor: Walter Vermeiren, Houthalen (BE)

(73) Assignee: TOTAL RESEARCH & TECHNOLOGY FELUY, Seneffe (BE)

(21) Appl. No.: 13/994,988

(22) PCT Filed: Dec. 15, 2011
(86) PCT No.: PCT/EP2011/072958

§ 371 Date: Oct. 10, 2013

(30) Foreign Application Priority Data
Dec. 17, 2010 (EP) 10195765.2

(51) Int. Cl. C12P 5/02; C07C 1/24; C08F 10/06

(52) U.S. Cl. CPC C12P 5/02; C08F 10/06; C07C 1/24
USPC 526/339; 526/348; 526/348.5; 526/348.2; 526/348.3; 585/16; 435/167

(57) ABSTRACT

A process for making propylene by dehydration of propanol can include gasifying a carbonaceous solid or liquid feedstock, or reforming a gaseous carbonaceous feedstock into synthesis gas. The synthesis gas can be fermented or co-fermented by means of a microorganism into propanol. The microorganism can be a wild strain having the natural capability to ferment synthesis gas into propanol. The microorganism can be a microorganism possessing the required nucleic acid sequence information to express the enzymes for the biosynthesis of C₃-oxygenates modified with the required nucleic acid sequence information to express the enzymes of the Wood-Ljungdahl pathway. The microorganism can be a microorganism possessing the required nucleic acid sequence information to express the enzymes of the Wood-Ljungdahl pathway, modified with the required nucleic acid sequence information to express the enzymes for the biosynthesis of C₃-oxygenates. The stream can be fractionated, purified, and then dehydrated at conditions effective to make propylene.

### FIELD OF THE INVENTION

The present invention relates to the production of propylene via dehydration of propanol on advantageously an acidic catalyst, whereby the propanol is obtained by metabolic conversion of synthesis gas, that is produced by gasification of biomass, waste, coal, liquid residu's, effluent gases from steel furnaces or by reforming of natural gas, into propanol (either n-propanol or isopropanol). The limited supply and increasing cost of crude oil has prompted the search for alternative processes for producing hydrocarbon products such as propylene. Propanol can be obtained by metabolic conversion of synthesis gas by microorganisms. Made up of organic matter from living organisms, biomass is the world's leading renewable energy source.

### BACKGROUND OF THE INVENTION

The production of propanols is limited compared to the production of propylene. In 2005, the world production of n-propanol was about 140 kta and of isopropanol more than 2000 kta whereas the world production of propylene exceeds 65000 kta.

Historically, isopropanol has been produced by hydration of propylene over acidic catalyst (Kirk-Othmer Encyclopedia of Chemical Technology, 2007 & Ullmann's Encyclopedia of Industrial Chemistry, 2002). In the indirect hydration, isopropyl sulfate esters are formed out of propylene and sulfuric acid, which is subsequently hydrolyzed into isopropanol. In the direct hydration, propylene is converted at high pressure and low temperature over an acid fixed bed catalyst. n-Propanol can be obtained by hydroformylation of ethylene via the intermediate propanal that is hydrogenated further in n-propanol. Typical conditions employed in the low pressure rhodium-substituted phosphine catalysed oxo process are 90-130° C. and less than 28 bars whereas the conditions for the cobalt catalysed oxo process are 110-180° C. and above 200 bars. The propanal is subsequently hydrogenated into n-propanol in the presence of excess hydrogen over a metallic catalyst, typically composed of copper, zinc, nickel and chromium compounds.

Propanol is also been produced by hydrogenation of acetone over metallic catalyst into isopropanol. Acetone is the byproduct in the phenol production from cumene. (Ullmann's Encyclopedia of Industrial Chemistry, 2002).

Aceton, as one of the three products in the ABE (Aceton-butanol-ethanol) fermentation technology based on sugars, could also be hydrogenated into isopropanol (“Bacterial acetone and butanol production by industrial fermentation in the Soviet Union: use of hydrolyzed agricultural waste for biorefinery”, Appl. Microbiol. Biotechnol., 71, p. 587-597, 2006; “History of the Acetone-Butanol-Ethanol Fermentation Industry in China: Development of Continuous Production Technology”, J. Mol. Microbiol. Biotechnol., 13, p. 12-14, 2007; “Acetone-butanol fermentation revisited”, Microbiol. Rev. 50, p. 484-524, 1986 and Jones, D., “Applied acetone-butanol fermentation”, In “Clostridia. Biotechnology and medical application”, p. 125-168, 2001, Wiley-VCH Verlag GmbH, Weinheim, Germany). The acetone-butanol-ethanol (ABE) fermentation by Clostridium acetobutylicum is one of the oldest known industrial fermentations. It was ranked second only to ethanol fermentation by yeast in its scale of production, and is one of the largest biotechnological processes ever known. However, since the 1950's industrial ABE fermentation has declined gradually, and almost all acetone is now produced via petrochemical routes. More recently, biochemical metabolic routes are being developed for making propanol from carbohydrates. US Patent 2009/0246842 describes a process for the fermentation of sugars into isopropanol.

Propanol can also be produced from synthesis gas (mixture of CO, H₂ and CO₂) by a catalytic process similar to Fischer-Tropsch, resulting in a mixture of higher alcohols, although often a preferential formation of propanol occurs (Applied Catalysis A, general, 186, p. 407, 1999 and Chemiker Zeitung, 106, p. 249, 1982). Still another route to obtain propanol, is the base-catalysed Guerbet condensation of methanol with ethanol (J. of Molecular Catalysis A: Chemical 200, 137, 2003, J. Chem. Soc. Chem. Commmun., 22, p. 1558, 1990 and Applied Biochemistry and Biotechnology, 113-116, p. 913, 2004).

Whereas in the past, propanols have been produced mostly out of ethylene or propylene, being high added value chemicals, there is a need to produce propanols from other carbon sources so that the respective propanols can be dehydrated into propylene.

Since many centuries, simple sugars are being fermented into ethanol with the help of sacharomycis cerevisae. The last decade's new routes starting from cellulose and hemicelluloses have been developed to ferment more complex carbohydrates into ethanol. Hereto, the carbohydrates need to be unlocked from the lignocellulosic biomass. Biomass consists approximately of 30% cellulose, 35% hemicelluloses and 25% lignin. The lignin fraction cannot be valorised as ethanol, as of its aromatic nature but can only be used as energy source which present in many cases an excess for running an industrial plant. Recently, more efficient routes that produce synthesis gas from carbon-containing materials and that subsequently is fermented into ethanol are being developed (“Bioconversion of synthesis gas into liquid or gaseous fuels”, K. Klasson, M. Ackerson, E. Clausen, J. Gaddy, Enzyme and Microbial Technology, 14(8), p. 602, 1992; “Fermentation of Biomass-Generated Producer Gas to Ethanol”, R. Datar, R. Shenkman, B. Cateni, R. Huhnke, R. Lewis, Biotechnology and Bioengineering, 86 (5), p. 587, 2004; “Microbiology of synthesis gas fermentation for biofuel production”, A. Hemstra, J. Sipma, A. Rinzema, A. Stams, Current Opinion in Biotechnology, 18, p. 200, 2007; “Old Acetogens, New Light”, H. Drake, A. Göβner, S. Daniel, Ann. N.Y. Acad. Sci. 1125: 100-128, 2008). Synthesis gas can be produced by gasification of the whole biomass without need to unlock certain fractions. Synthesis gas can also be produced from other feedstock via gasification: (i) coal, (ii) municipal waste (iii) plastic waste, (iv) petcoke and (v) liquid residu's from refineries or from the paper industry (black liquor). Synthesis gas can also be produced from natural gas via steamreforming or autothermal reforming (partial oxidation). For conventional methanol synthesis, higher alcohol synthesis or Fischer-Tropsch a ratio of hydrogen to carbonmonoxide of about 2 is required. In case of gasification of hydrogen-poor feedstock this ratio will be below 1 and hence a watergas shift (CO+H₂O→CO₂+H₂) is required to adjust the ratio. The biochemical pathway to transform synthesis gas into ethanol is much less stringent regarding the hydrogen to carbonmonoxide ratio.

The biochemical pathway of synthesis gas conversion is described by the Wood-Ljundahl Pathway. Fermentation of syngas offers several advantages such as high specificity of biocatalysts, lower energy costs (because of low pressure and low temperature bioconversion conditions), greater resistance to biocatalyst poisoning and nearly no constraint for a preset H₂ to CO ratio (“Reactor design issues for synthesis-gas fermentations” M. Bredwell, P. Srivastava, R. Worden, Biotechnology Progress 15, 834-844, 1999; “Biological conversion of synthesis gas into fuels”, K. Klasson, C. Ackerson, E. Clausen, J. Gaddy, International Journal of Hydrogen Energy 17, p. 281, 1992). Acetogens are a group of anaerobic bacteria able to convert syngas components, like CO, CO₂ and H₂ to acetate via the reductive acetyl-CoA or the Wood-Ljungdahl pathway.

Several anaerobic bacteria have been isolated that have the ability to ferment syngas to ethanol, acetic acid and other useful end products. Clostridium ljungdahlii and Clostridium autoethanogenum, were two of the first known organisms to convert CO, CO₂ and H₂ to ethanol and acetic acid. Commonly known as acetogens, these microorganisms have the ability to reduce CO₂ to acetate in order to produce required energy and to produce cell mass. The overall stoichiometry for the synthesis of ethanol using three different combinations of syngas components is as follows (J. Vega, S. Prieto, B. Elmore, E. Clausen, J. Gaddy, “The Biological Production of Ethanol from Synthesis Gas”, Applied Biochemistry and Biotechnology, 20-1, p. 781, 1989):

6CO+3H₂O→CH₃CH₂OH+4CO₂

2CO₂+6H₂→CH₃CH₂OH+3H₂O

6CO+6H₂→2CH₃CH₂OH+2CO₂

Acetogenic bacteria are obligate anaerobes that utilize the acetyl-CoA pathway as their predominant mechanism for the reductive synthesis of acetyl-CoA from CO₂ (Drake, H. L. (1994). Acetogenesis. New York: Chapman & Hall). This group of microorganisms is even more versatile in the sense that they can use simple gases like CO₂/H₂ and CO as well as sugars, carboxylic acids, alcohols and aminoacids.

Clostridium ljungdahlii, one of the first autotrophic microorganism known to ferment synthesis gas to ethanol was isolated in 1987, as an acetogen favours the production of acetate during its active growth phase (acetogenesis)) while ethanol is produced primarily as a non-growth-related product (solventogenesis) (“Biological conversion of synthesis gas into fuels”, K. Klasson, C. Ackerson, E. Clausen, J. Gaddy, International Journal of Hydrogen Energy 17, p. 281, 1992). Eubacterium limosum is an acetogen, isolated from habitats like the human intestine, rumen, sewage and soil, exhibits high growth rate under high CO concentrations producing acetate, ethanol, butyrate and isobutyrate (I. Chang, B. Kim, R. Lovitt, J. Bang, “Effect of CO partial pressure on cell-recycled continuous CO fermentation by Eubacterium limosum KIST612”, Process Biochemistry, 37(4), p. 411, 2001).

Peptostreptococcus productus is a mesophilic, gram-positive anaerobic coccus, found in the human bowel and is capable of metabolizing CO₂/H₂ or CO to produce acetate (W. Lorowitz, M. Bryant, “Peptostreptococcus productus Strain That Grows Rapidly with CO as the Energy-Source”, Applied and Environmental Microbiology, 47(5), p. 961, 1984).

Clostridium autoethanogenum is a strictly anaerobic, gram-positive, spore-forming, rod-like, motile bacterium which metabolizes CO to form ethanol, acetate and CO₂ as end products, beside it ability to use CO₂ and H₂, pyruvate, xylose, arabinose, fructose, rhamnose and L-glutamate as substrates (J. Abrini, H. Naveau, E. Nyns), “Clostridium autoethanogenum, Sp-Nov, an Anaerobic Bacterium That Produces Ethanol from Carbon-Monoxide”, Archives of Microbiology, 161(4), p. 345, 1994).

Clostridium carboxidivorans P7 is a solvent-producing anaerobe, which was isolated from the sediment of an agricultural settling lagoon. It is motile, gram-positive, spore-forming and primarily acetogenic, forming acetate, ethanol, butyrate, and butanol as end-products. (J. Liou, D. Balkwill, G. Drake, R. Tanner, “Clostridium carboxidivorans sp. nov., a solvent-producing clostridium isolated from an agricultural settling lagoon, and reclassification of the acetogen Clostridium scatologenes strain SL1 as Clostridium drakei sp. nov.”, International Journal of Systematic and Evolutionary Microbiology, 55(5), p. 2085, 2005).

Acetogens are obligate anaerobic bacteria that use the reductive acetyl-CoA pathway as their predominant (i) mechanism for the reductive synthesis of acetyl-CoA from CO₂, (ii) terminal electron-accepting, energy-conserving process, and (iii) mechanism for the synthesis of cell carbon from CO₂″ (Drake, H. L. (1994). Acetogenesis. New York: Chapman & Hall). Like other anaerobes, acetogens require a terminal electron acceptor different from oxygen. In the acetyl-CoA pathway, CO₂ serves as an electron acceptor and H₂ serves as the electron donor. The synthesis of acetyl-CoA from CO₂ and H₂ requires an 8-electron reduction of CO₂ involving the following three steps:

Formation of the carbonyl precursor of acetyl-CoA
Formation of the methyl precursor of acetyl-CoA
Condensation of the above two precursors to form acetyl-CoA.

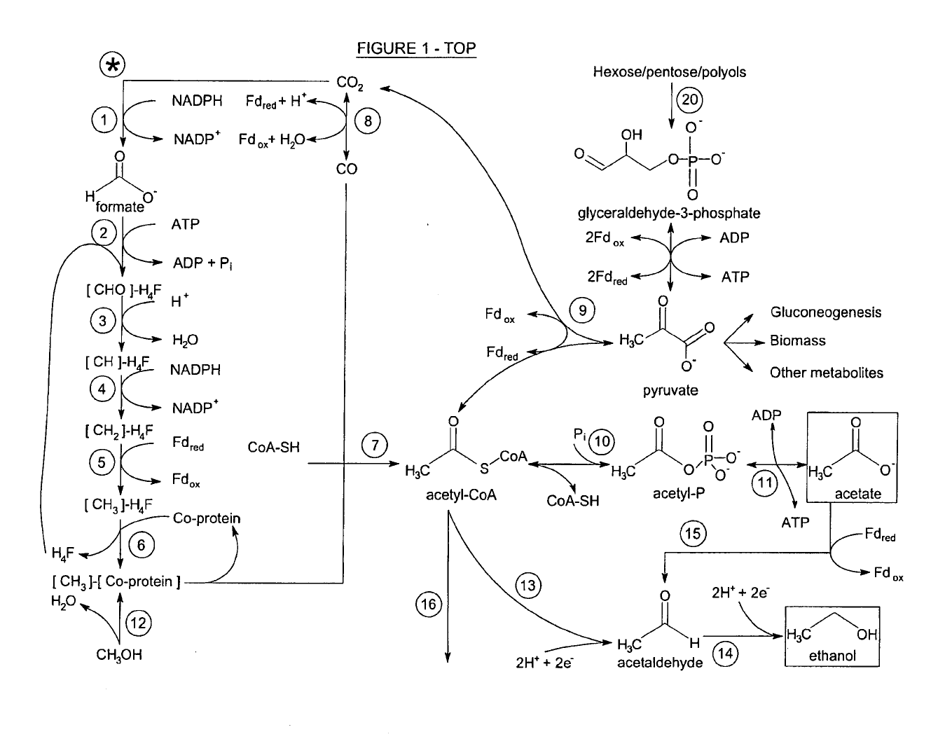

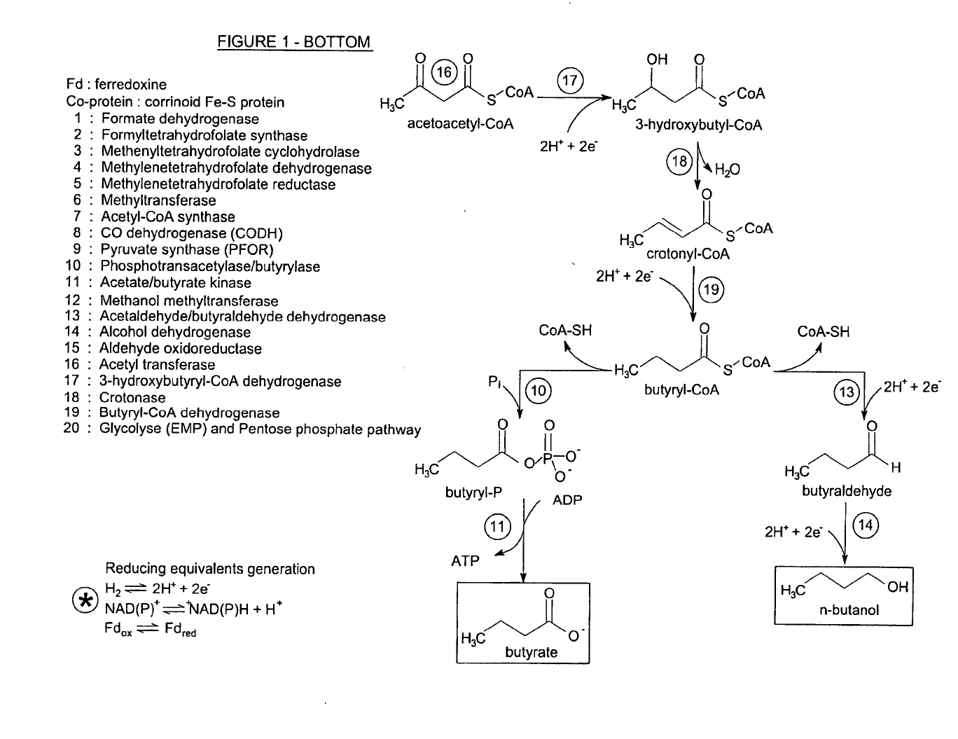

### The Methyl Branch of the Acetyl-CoA Pathway (see FIG. 1)

This part of the pathway results in the formation of the methyl-corrinoid protein that combines with the product of the carbonyl branch, to form acetyl-CoA. In the first step of this branch, CO₂ is reduced to formate (HCOO) as shown in the following equation:

CO₂+2[H]→HCOO⁻+H⁺

This reversible reaction is catalyzed by the enzyme formate dehydrogenase (FDH). Ferredoxin is the most commonly employed electron acceptor, among acetogens, NADH often acts as the electron donor. For acetogens grown on CO, Ljungdahl suggested that CO must first be oxidised to CO₂ by the enzyme carbon monoxide dehydrogenase (CODH) and subsequently reduced to formate by FDH (L. Ljungdahl, “The autotrophic pathway of acetate synthesis in acetogenic bacteria”, Annual Review of Microbiology, 40, 415, 1986).

Formate is activated with tetrahydrofolate (THF) to form 10-formyl-THF by the enzyme formyl-THF synthetase in an ATP-dependent condensation. This bound formyl group is then reduced by a series of 3 enzymes to a bound methyl group (methyl-THF). In the final step of this branch, the methyl group is transferred to a corrinoid-containing protein [Co]-protein.

### The Carbonyl Branch (see FIG. 1)

This branch of the pathway results in the formation of a bound carbonyl group which is then merged with the bound methyl group formed in the methyl branch to form acetyl-CoA. Carbon monoxide dehydrogenase (CODH) plays a very essential role with a double functionality. First, it catalyzes the oxidation of CO to CO₂, the reduction of CO₂ to bound carbonyl, finally mediating the synthesis of acetyl-CoA from the methyl and carbonyl groups. For the latter reason, CODH is also known as acetyl-CoA synthase. In the carbonyl branch, CO₂ is first reduced to [CO] ([ ] indicates that carbon monoxide is enzyme-bound) as follows:

CO₂+2[H]←→[CO]+H₂O

The bound carbonyl moiety is condensed with the bound methyl moiety from the methyl branch to form a bound acetyl-CODH moiety. In the final step, CODH condenses the bound acetyl with free coenzyme A to form acetyl-CoA, as follows:

CH₃—[Co]-protein+[CO]+HS-CoA→Acetyl-CoA+[Co]-protein

Hydrogenase enzymes are used by microorganisms either to dispose of electrons accumulated during fermentation via hydrogen formation, or hydrogen uptake and oxidation to produce energy. The reaction involving hydrogen is a reversible reaction catalysed by hydrogenase:

H₂←→2H⁺+2e⁻

The CODH enzyme works in combination with hydrogenase to form the carbonyl precursor of acetyl-CoA (L. Ljungdahl, “The autotrophic pathway of acetate synthesis in acetogenic bacteria”, Annual Review of Microbiology, 40, 415, 1986).

Acetyl-CoA is a central intermediate in the metabolic pathway of acetogens as it is a versatile precursor of alcohols, carboxylic acids, diacids, hydroxyacids, diols, lipids, amino acids, nucleotides and carbohydrates (Ljungdahl, L., “The autotrophic pathway of acetate synthesis in acetogenic bacteria”, Annual Review of Microbiology, 40, p. 415-450, 1986.): (i) for cellular material, formed via the anabolic pathway, in which acetyl-CoA is reductively carboxylated into pyruvate by the enzyme pyruvate synthase (Diekert, G., “Metabolism of Homoacetogens”, Antonie Van Leeuwenhoek International Journal of General and Molecular Microbiology, 66(1-3), p. 209-221, 1994) that is subsequently converted to phosphoenolpyruvate (PEP) which is an intermediate in the conversion to biomass and (ii) for energy conservation, acetyl-CoA goes through the catabolic pathway in order to make ATP. Beside the essential intermediate PEP that is at the basis of many biochemical pathways, acetyl-CoA can condense further to longer chain hydrocarbyl moieties.

In case of homoacetogens the acetyl-CoA is converted to acetate catalysed by Phosphotransacetylase and acetate kinase while producing ATP by substrate-level phosphorylation:

Acetyl-CoA+Pi←→Acetyl-Phosphate+CoA

Acetyl-Phosphate+ADP←→Acetate+ATP

In the first reaction, the CoA unit is removed from the acetyl-CoA and a phosphate group is added by the enzyme phosphotransacetylase, resulting in the formation of acetyl-phosphate. In the second reaction, the acetyl phosphate is converted to acetate while a molecule of adenosine diphosphate (ADP) is phosphorylated to form ATP. This part of energy-conservation pathway is usually favoured over the alcohol forming pathway during its exponential growth phase of the microorganism as it provides the cell with energy in the form of ATP, known as the acidogenic phase of the metabolism, which also results in a decrease in pH of the medium due to acid production. The second phase of the fermentation is the solventogenic phase, in which mainly ethanol is produced:

Acetyl-CoA+NADH+H⁺←→Acetaldehyde+CoA-H+NAD⁺

Acetaldehyde+NADH+H⁺←→Ethanol+NAD⁺

In the solventogenic branch of the pathway, the microorganism utilizes NADH as the reducing potential to first form acetaldehyde by the enzyme acetaldehyde dehydrogenase, followed by further reduction to ethanol by the enzyme alcohol dehydrogenase.

Many acetogens (Clostridium acetobutylicum) have the ability to produce 4-carbon products like butanol and butyric acid by condensation of 2 molecules of acetyl-CoA to form acetoacetyl-CoA that is further isomerised into butyryl-CoA. Analogously to acetyl-CoA, subsequent transformation into butyric acid produces ATP while the formation of butanol results in the consumption of reducing equivalents:

Butyryl-CoA+Pi←→Butyryl-Phosphate+CoA

Butyryl-Phosphate+ADP←→Butyrate+ATP

Butyryl-CoA+NADH+H⁺←→Butyraldehyde+CoA-H+NAD⁺

Butyraldehyde+NADH+H⁺←→Butanol+NAD⁺

### Pathways for the Production of Oxygenates Having Three Carbons:

### 1. Propionic Acid Production

Propionibacterium species (Propionibacterium acidipropionici, Propionibacterium acnes, Propionibacterium cyclohexanicum Propionibacterium freudenreichii, Propionibacterium freudenreichii shermanii) and several other anaerobic bacteria such as Desulfobulbus propionicus, Pectinatus frisingensis, Pelobacter propionicus, Veillonella, Selenomonas, Fusobacterium and Clostridium, in particular Clostridium propionicum, produce propionic acid as a main fermentation product (Playne M., “Propionic and butyric acids”, In: Moo-Young M, editor. Comprehensive biotechnology, New York: Pergamon Press, vol 3, p 731-759, 1985; Seshadri N, Mukhopadhyay S., “Influence of environmental parameters on propionic acid upstream bioprocessing by Propionibacterium acidi-propionici”, J. Biotechnology 29, p. 321-328, 1993). In swiss-type cheeses, propionibacteria consume lactate and produce propionic acid, acetic acid, and CO₂. In general, a broad range of substrates can be converted into propionic acid, like glucose, lactose, sucrose, xylose, glycerol and lactate. Propionibacteria are Gram-positive, non-motile, non-sporulating, short-rodshaped, mesophilic anaerobes. The genus of Propionibacterium, belonging to the class of high G+C actinobacteria is divided into two groups: the “cutaneous” and the “dairy” Propionibacteria, based on their habitat (Stackebrandt, E., Cummins, C., Johnson, J., “The Genus Propionibacterium”, in The Prokaryotes, E. Balows, H. Truper, M. Dworkin, W. Harder, K. Scheifer, eds., 2006).

a. Dicarboxylic Pathway

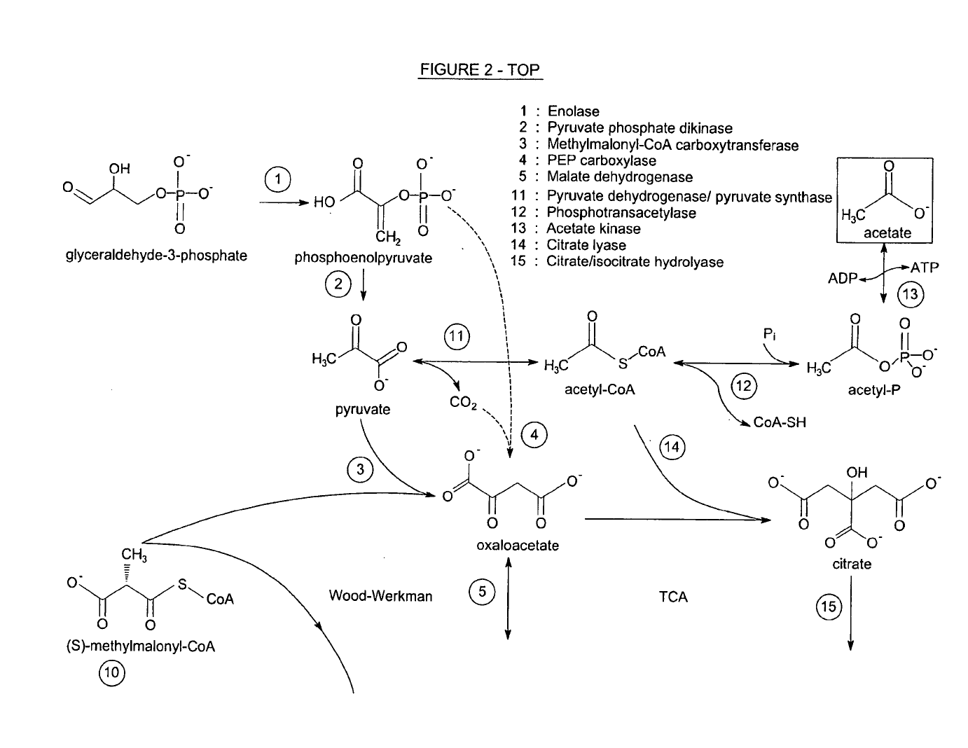

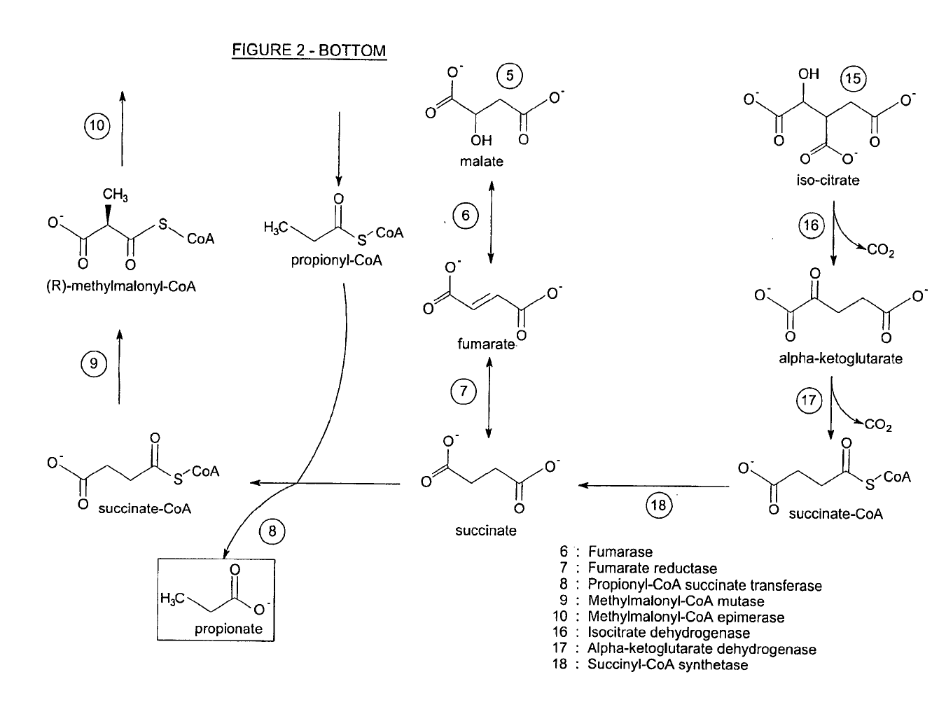

Propionibacteria convert carbon sources to produce propionic acid as a main product via the mainly dicarboxylic acid pathway (also called the Wood-Werkman cycle or the methyl-malonyl-CoA pathway), as shown in FIG. 2. Glycolysis pathway catabolyses glucose into phosphoenolpyruvate (PEP), an energy-rich metabolite. Two alternative glycolysis pathways exist: Embden-Meyerhorf-Parnaz (EMP) pathway and Hexose Monophosphate (HMP) pathway. In the EMP pathway, 1 mole of glucose is converted into 2 moles of PEP and 2 moles of NADH, while in the HMP pathway 1 mole of glucose provides 5/3 moles of PEP and 11/3 moles of NADH. PEP is further converted into two possible intermediates, pyruvate and oxaloacetate. The majority of PEP is converted into pyruvate whereas the remaining PEP is converted into oxaloacetate. For pyruvate production, 1 mole of PEP is converted into 1 mole of pyruvate and 1 mole of ATP obtained from a transfer of one phosphoryl moiety from PEP to ADP. The total ATP obtained from the EMP and HMP pathways per mole of glucose is 2 and 5/3 moles, respectively. Glycolysis via the EMP pathway provides a lower amount of NADH (EMP: HMP=2: 11/3) but a higher amount of ATP (EMP: HMP=2:5/3). The ratio of EMP to HMP pathway contribution in glycolysis is dependent on propionibacterium species, substrates and fermentation conditions. At the pyruvate node, pyruvate is directed toward three main pathways. Most of pyruvate is converted into propionic acid via the Wood-Werkman cycle. Some of pyruvate converts into acetate while some is incorporated into biomass. In the propionate formation pathway, pyruvate enters the Wood-Werkman cycle, via a transcarboxylation of a carboxyl-moiety from methylmalonyl-CoA to pyruvate, catalysed by oxaloacetate transcarboxylase in a coupled reaction of pyruvate to oxaloacetate and methylmalonyl CoA to propionyl CoA. In this coupled reaction, the carboxyl group transferred from methylmalonyl CoA to pyruvate to form propionyl CoA and oxaloacetate is never released from the reaction or no exchange between this carboxyl group with the dissolved CO₂ in the fermentation broth is observed (Wood H G., “Metabolic cycles in the fermentation of propionic acid”, in Current Topics in Cellular regulation, Estabrook and Srera R W, eds., New York: Academic Press. vol 18, p 225-287, 1981). Because of this transcarboxylation reaction, CO₂ fixation is minimal and only used to produce catalytic amounts of oxaloacetate to reinitiate the cycle when for instance succinate accumulates as end-product. Under such circumstances, oxaloacetate is generated by condensation of CO₂ with phosphoenolpyruvate catalysed by a PEP carboxylase. Susequently, oxaloacetate is converted into malate by malate dehydrogenase, malate into fumarate by fumarase and further fumarate to succinate, catalyzed by succinate dehydrogenase. After that succinate is converted into succinyl-CoA, which is then converted into methylmalonyl-CoA. Methylmalonyl-CoA is converted into propionyl-CoA by oxaloacetate transcarboxylase. At the end of the cycle, propionyl-CoA is converted into propionate along with a coupled reaction of succinate to succinyl-CoA, catalysed by propionyl-CoA: succinate transferase. After 1 mole of pyruvate enters the Wood-Werkman cycle, 1 mole of propionate, 2 moles of NAD⁺, and 1 mole of ATP are generated. Beside propionic acid as main fermentation product, produced in the Wood-Werkman cycle, also NAD⁺ regeneration for glycolysis occurs in this cycle.

In acetate branch pathway, pyruvate converts to acetyl-CoA and CO₂, catalyzed by pyruvate dehydrogenase complex. Acetyl-CoA is converted into acetyl-phosphate by phosphotransacetylase and further acetyl-phosphate to acetate, catalyzed by acetate kinase. In the acetate branch pathway, 1 mole of acetate, CO₂, NADH, and ATP are obtained from 1 mole of pyruvate. Propionic acid production is usually accompanied by the acetate formation as a major ATP production route supplying energy for cellular metabolism.

The following equations represent a theoretical formulation of propionic acid fermentation from glucose or lactate (P. Piveteau, Lait, 79, p. 23, 1999):

1.5glucose+6Pᵢ+6ADP→2propionate+acetate+CO₂+2H₂O+6ATP

3lactic acid+3Pᵢ+3ADP→2propionate+acetate+CO₂+2H₂O+3ATP

According to these equations, the theoretical maximum yield from glucose is 66.7 C-mole % or 54.8 wt % of propionic acid, 22.2 C-mole % or 22 wt % of acetic acid, 11.1 C-mole % or 17 wt % of CO₂. The theoretically propionic acid to acetic acid (P/A) molar ratio is 2:1.

A shift in the metabolic pathway towards the production of propionic acid can be accomplished by using carbon sources with higher reductive level (shift from heterofermentative to homofermentative acid production). A higher reductive level of substrate can cause significant increase in the P/A ratio due to the intracellular NADH/NAD⁺ balance. A better efficiency of propionic acid production from glycerol could be expected because of its higher reduction level compared to conventional substrates. Effectively, a propionic acid yield of 84.4 C-mole % and a low acetic acid production (P/A molar ratio reaching 37) have been obtained from glycerol with P. acidipropionici (Barbirato, F., Chedaille, D. and Bories, A., “Propionic acid fermentation from glycerol: comparison with conventional substrates”, Appl Microbiol Biotechnol, 47, p. 441-446, 1997). This strain also produces some propanol from glycerol, indicating that when the substrate has a higher reduction level also products with a higher reduction level can be produced because of the better NADH/NAD⁺ balance.

Glycerol→propionate+1H₂O

Himmi et. al. compared the fermentation of glycerol and glucose and product formation for P. acidipropionici and P. freudenreichii ssp. shermanii. Fermentation end-products were propionic acid as the major product, acetic acid as the main byproduct and two minor metabolites, n-propanol and succinic acid. The yield of propionic acid was up to 79 C-mole % (64 wt %) with glycerol as the carbon source (Himmi, E. H., Bories, A., Boussaid, A. and Hassani, L., “Propionic acid fermentation of glycerol and glucose by Propionibacterium acidipropionici and Propionibacterium freudenreichii ssp. Shermanii”, Appl Microbiol Biotechnol, 53, p. 435-440, 2000). Rumen microorganisms that ferment lactate via the dicarboxylic acid pathway, produce more propionate relative to acetate when hydrogen is added (M. Schulmanda and D. Valentino, “Factors Influencing Rumen Fermentation: Effect of Hydrogen on formation of Propionate”, Journal of Dairy Science, vol. 59 (8), p. 1444-1451, 1976). Acetic acid was almost eliminated when a high H₂ pressure was applied during the fermentation with Propionispira arboris containing hydrogenase (Thompson T. E, Conrad R, Zeikus J. G., “Regulation of carbon and electron flow in Propionispira arboris: Physiological function of hydrogenase and its role in homopropionate formation”, FEMS Microbiol Lett 22, p. 265-271, 1984 and U.S. Pat. No. 4,732,855). According to the Wood-Werkman cycle, endogenous CO₂ is released with acetic acid formation by Propionibacteria from glucose, lactose, or lactate fermentation (Deborde C., Boyaval P. 2000, Interactions between pyruvate and lactate metabolism in Propionibacterium freudenreichii subsp. shermanii: In vivo ¹³C nuclear magnetic resonance studies, Appl Environ Microbiol 66: 2012-2020). CO₂ can be fixed in Propionibacteria to form oxaloactate from PEP catalyzed by PEP carboxylase and then lead to succinate generation. Based on the metabolic pathway (Wood-Werkman cycle), CO₂ (HCO₃⁻) is required to convert phosphoenolypyruvate (PEP) into oxaloacetate by the enzyme phosphoenolypyruvate carboxylase. Through several sequential reactions, oxaloacetate is finally converted to propionic acid. In case of glycerol as substrate, nearly no acetate and hence CO₂ is produced. Applying an exogenous CO₂ pressure during fermentation has an positive effect on metabolite production rate and in particular a higher succinate accumulation thanks to the higher PEP carboxylation activity (“Effect of carbon dioxide on propionic acid productivity from glycerol by Propionibacterium acidipropionici”, An Zhang and Shang-Tian Yang, SIM annual meeting and Exhibition, San Diego, 2008).

Most propionic acid producing bacteria have the enzymes of the tricarboxylic acid cycle (TCA) which explain the variable P/A ratios for different strains. Some of the acetyl-CoA can be utilized in the TCA cycle by condensation with pyruvate into citrate (see FIG. 2). The end result is that more CO₂ is produced in the TCA cycle through the decarboxylations and less acetate is secreted. P/A ratios from 2.1 to 14.7 and CO₂/acetate ratio from 1.0 to 6.3 have been reported from glucose (Wood H G., “Metabolic cycles in the fermentation of propionic acid”, in Current Topics in Cellular regulation, Estabrook and Srera R W, eds., New York: Academic Press. vol 18, p 225-287, 1981).

Pelobacter propionicus, using the dicarboxylic acid pathway, has been show to grow on ethanol as substrate while producing propionate in presence of CO₂ (Schink, B., Kremer, D. and Hansen, T., “Pathway of propionate formation from ethanol in Pelobacter propionicus”, Arch. Microbiol. 147, 321-327, 1987 and S. Seeliger, P. Janssen, B. Schink, “Energetics and kinetics of lactate fermentation to acetate and propionate via methylmalonyl-CoA or acrylyl-CoA”, FEMS Microbiology Letters, 211, pp. 65-70, 2002). When ethanol is fed together with CO₂ and hydrogen, significant amounts of propanol are produced. Ethanol is converted into acetyl-CoA (via acetaldehyde) while producing electrons for the carboxylation of acetyl-CoA into pyruvate, catalysed by pyruvate synthase. Combined with the dicarboxylic acid pathway propionate is produced from ethanol and CO₂ (Schink et al., 1987).

3ethanol+2HCO₃⁻→2propionate⁻+acetate⁻+H⁺+3H₂O

Pelobacter propionicus is not able to reductively convert acetate and CO₂ into propionate whereas Desulfobulbus propionicus does make propionate from acetate and CO₂ (Schink et al., 1987).

acetate⁻+HCO₃⁻+3H₂→propionate⁻+3H₂O

b. Acrylate Pathway

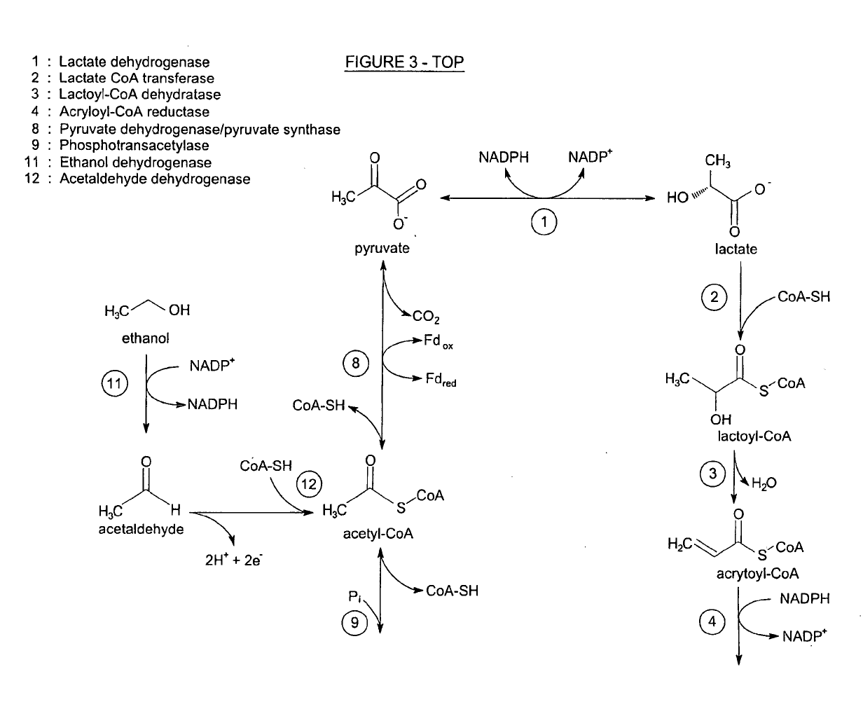

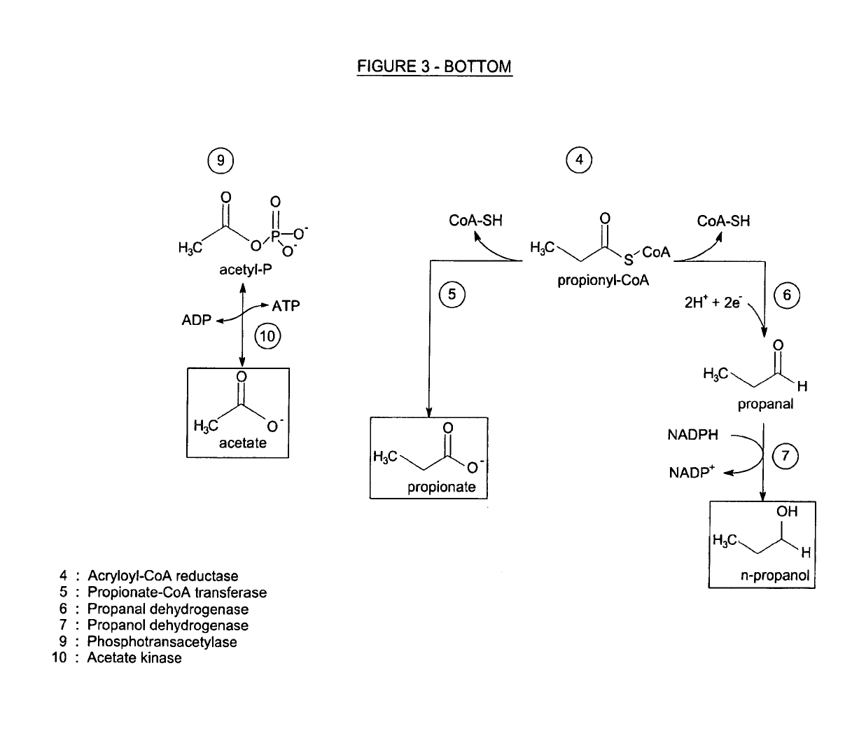

Though many bacteria can ferment a variety of substrates anaerobically into lactate as end product, some can further reduce the lactate into propionate, like Clostrium propionicum, Clostrium neopropionicum, Megasphaera elsdenii and Prevotella ruminicola (P. Boyaval, C. Corre, “Production of propionic acid”, Lait, 75, 453-461, 1995) by using the acryloyl-CoA pathway (see FIG. 3). Several substrates (sugars, ethanol and some aminoacids) that can be converted into pyruvate as intermediate can be further reduced into propionate as main product with acetate and butyrate as co-product. The key reaction is the lactoyl-CoA dehydration into acryloyl-CoA that is subsequently reduced to propionyl-CoA. The electrons for this reduction are provided by the oxidation of pyruvate/lactate into acetate and CO₂ (G. Gottschalk, “Bacterial Metabolism”, 2^{nd} ed., Springer, New York, 1986).

Clostridium neopropionicum (strain X4), using the acrylate pathway, is able to convert ethanol and CO₂ into acetate, propionate and some propanol (J. Tholozan, J. Touzel, E. Samain, J. Grivet, G. Prensier and G. Albagnac, “Clostridium neopropionicum sp. Nov., a strict anaerobic bacterium fermenting ethanol to propionate through acrylate pathway”, Arch. Microbiol., 157, p. 249-257, 1992). As for the dicarboxylic acid pathway, the intermediate acetyl-CoA produced from the substrate ethanol is linked to the acrylate pathway via the pyruvate synthase that converts acetyl-CoA into pyruvate by carboxylation with CO₂.

Recently, an alternative route leading to acryloyl-CoA consists in the conversion of acetyl-CoA into malonyl-CoA by carboxylation with CO₂. The malonyl-CoA is further converted into acryloyl-CoA via four steps implicating malonate-semialdehyde, hydroxypropanoate, hydroxypropanoyl-CoA and finally acryloyl-CoA. Acryloyl-CoA produced by this pathway is subsequently reduced to propionyl-CoA similarly to the reactions leading to acryloyl-CoA by dehydratation of lactoyl-CoA (J. Zarzycki, “Identifying the missins steps of the autotrophic 3-hydroxypropionate CO2 fixation cycle in Chloroflexus aurantiacus, PNAS, 106(50), p. 21317, 2009; I. Berg, “A 3-hydroxypropionate/4-hydroxybutyrate autotrophic carbon dioxide assimilation pathway in archaea, Science, 318, p. 1782, 2007).

### 2. Aceton/Isopropanol Production

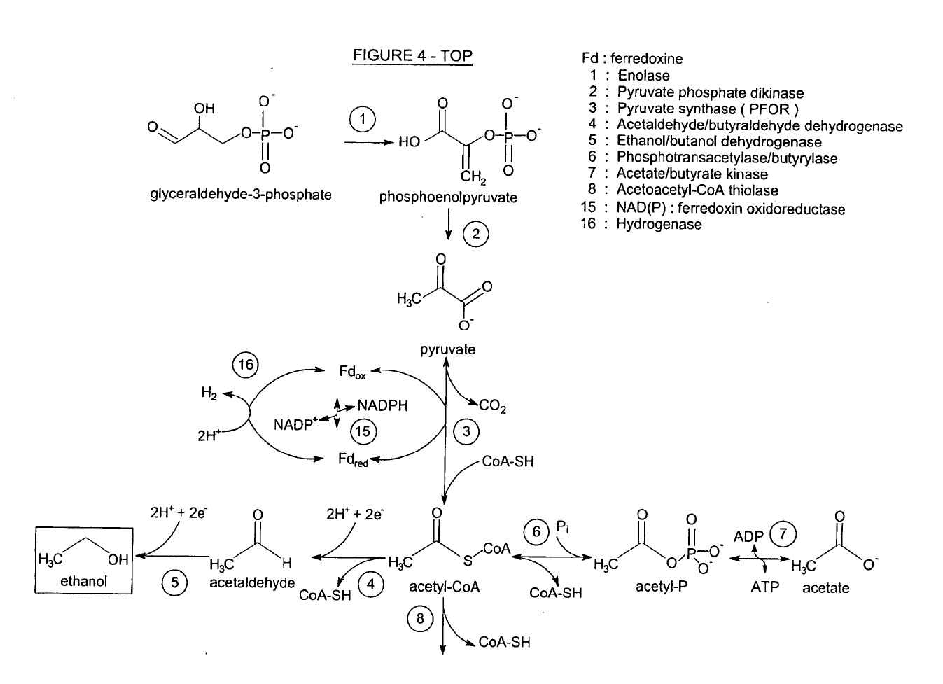

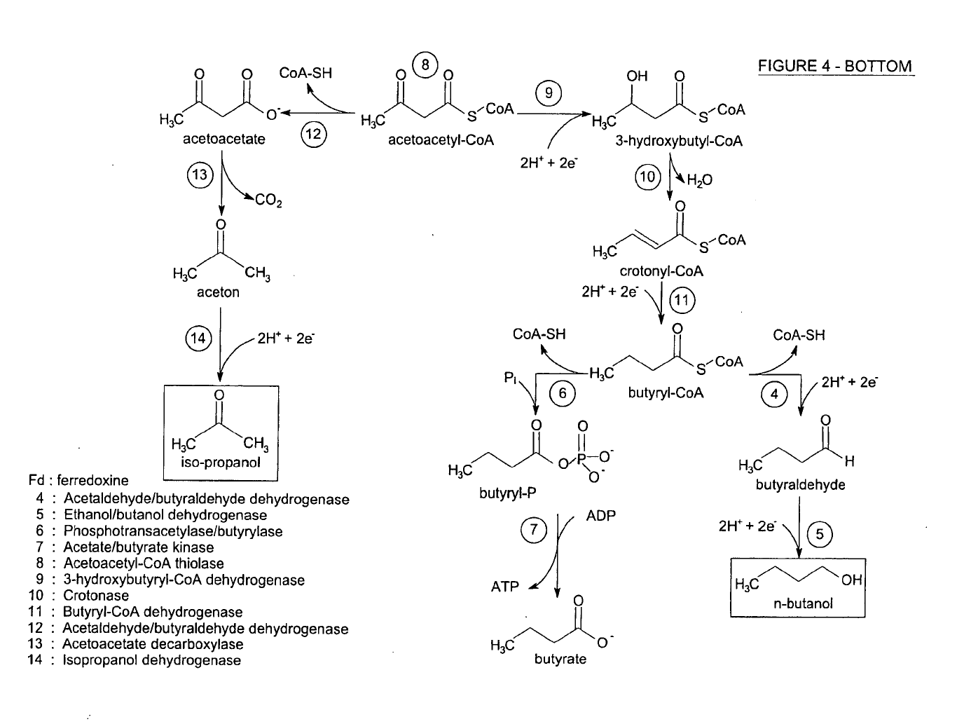

Members of the Clostridium, Butyrivibrio, Bacillus, and other less-well defined flora of anaerobic digestion systems produce butyric acid, butanol, acetone, isopropanol, or 2,3-butanediol (A. Moat, J. Foster & M. Spector, “Microbial Physiology”, 4th Ed., Wiley-Liss, 2002). Hydrogen, carbon dioxide, acetate, and ethanol are concomitantly produced in minor amounts during the fermentation. Clostridium acetobutylicum utilizes the EMP pathway for glucose catabolism with the formation of ethanol, carbon dioxide, hydrogen, acetone, isopropanol, butyrate, and butanol from pyruvate via acetyl-CoA as shown in FIG. 4 (D. Jones and D. Woods, “Acetone-Butanol Fermentation Revisited”, Microbiological Reviews, p. 484-524, 1986; R. Gheshlaghi, J. Scharer, M. Moo-Young, C. Chou, “Metabolic Pathways of Clostridia for producing butanol”, Biotechnology Advances, 27, p. 764, 2009).

Strains such as Clostridium acetobutylicum, Clostridium beijerinckii, Clostridium saccharobutylicum, and Clostridium saccharoperbutylacetonicum produce butanol in high concentration with acetone (or isopropanol) and ethanol. During growth, the organism first forms acetate and butyrate (acidogenic phase), disposing the excess electrons by reducing H⁺ to H₂. As the pH drops, due to the accumulation of acids, the culture is entering the stationary phase, there is a metabolic shift to solvent production (solvetogenic phase). Acetyl-CoA undergoes condensation to form acetoacetate, which may be reduced to butyrate and butanol or cleaved via decarboxylation to acetone with concomitant production of CO₂. Acetone may be further reduced to isopropanol. Acetyl-CoA may also be reduced to acetaldehyde and ethanol. During the solventogenic phase, sugars are fermented directly to solvents, while the present acidic products are also converted to solvents. Acetate and butyrate are activated to acetyl-CoA and butyryl-CoA through the reactions catalyzed by acetoacetyl-CoA:acetate-CoA transferase or kinase and phosphotransacetylase. These acyl-CoA's are reduced to ethanol and butanol by aldehyde dehydrogenase and alcohol dehydrogenase. Acetone can in some species further be reduced to isopropanol, like in Clostridium beijerinckii, since the alcohol dehydrogenase is receptive for different substrates, not only for aldehydes but also for ketones (A. Ismaierl, C. Zhu, G. Colby, J. Chen, “Purification and characterization of a primary-secondary alcohol dehydrogenase from two strain of Clostrium beijerinckii”, J. Bacterial., 175, p. 5097, 1993; J. Chen and S. Hiu, “Acetone-butanol-isopropanol production by Clostridium Beijerinckii” Biotechnology Letters, Vol 8(5), p. 371-376, 1986; H. George, J. Jonhson, W. Moore, L. Holdeman, J. Chen, “Acetone, Isopropanol, and Butanol Production by Clostridium beijerinckii (syn. Clostridium butylicum) and Clostridium aurantibutyricum”, Applied and Environmental Microbiology, p. 1160-1163, 1983). The decarboxylation of acetoacetate results in a sharp decrease in the number of electron (H⁺+e) acceptors available as CO₂ is expelled. Acetyl-CoA and acetone can act as electron acceptor, giving rise to ethanol or isopropanol. For the onset of solventogenesis, electron flux as well as carbon flux is redirected in order to preserve the oxido-reductive balance. More electrons are needed to produce solvents molecules (butanol, isopropanol and ethanol) than for the corresponding acids. The excess electrons that were used to reduce H⁺ to H₂ during the acidogenic phase are used by aldehyde dehydrogenase and alcohol dehydrogenase during the solventogenic phase. In the solvent-producing clostridia, NAD(P)⁺:ferredoxin oxidoreductase is active, exchanging electrons between NAD(P)⁺ and ferredoxin.

Fd_{red}+NAD(P)⁺+H⁺Fdₒₓ+NAD(P)H

During the acidogenic phase this enzyme is active to reduce ferredoxin while oxidizing NAD(P)H, and catalyzes the reverse reaction during solventogenesis. The regeneration of NAD(P)⁺ is required for the oxidation of glyceraldehydes-3-phosphate and prevents hence the accumulation of NAD(P)H. H₂ produced during acidogenesis is taken up by the bacteria for solvent production. Solventogenic clostridia have a H₂-producing hydrogenase as well as an uptake hydrogenase.

In clostridia and other anaerobes, hydrogen is formed through a pyruvate:ferredoxin (Fd) oxidoreductase without the intermediary production of formate. The reduced ferredoxin is converted to hydrogen by hydrogenase:

pyruvate+Fdₒₓ←→acetyl-CoA+CO₂+Fd_{red}+H⁺

Fd_{red}+2H⁺←→H₂+Fdₒₓ

Hydrogenase competes with ferredoxin:NAD(P) reductase to oxidize the reduced ferredoxin. The hydrogen evolution rate in the acidogenic phase is significantly higher. The flow of electrons from NADH to Fd_{red} and to H₂ explains why these organisms produce large quantities of H₂. During the Solventogenic phase more NAD(P)H is diverted to the formation of solvent alcohols by reduction of the corresponding acids. Several methods have been developed in order to reduce the iron-sulfide based hydrogenase activity and hence reducing the formation of hydrogen for the benefit of further reducing the acids into alcohols: external hydrogen pressure addition, carbonmonoxide addition that inhibits the iron-based hydrogenase activity, growth under iron-limiting conditions and co-fermenting highly reduced substrates like glycerol (see Gheshlaghi et al. and Jones et al.). Under such conditions the flow of electrons from reduced ferredoxin to molecular hydrogen via the hydrogenase system is inhibited and shifted to the generation of NAD(P)H via the action of the appropriate ferredoxin oxidoreductase, resulting in an increase in the production of butanol and ethanol. Often under reduced activity of the hydrogenase, also less aceton/isopropanol is produced as the formation of aceton by decarboxylation of acetoacetate lowers the amount of available electron acceptors.

Recently, E. Coli has been engineered with a specific pathway for the production of isopropanol from glucose by introducing acetyl-CoA acetyltransferase, acetoacetyl-CoA transferase, acetoacetate decarboxylase and alcohol dehydrogenase from the appropriate Clostrium species (T. Hanai, “Engineered Synthetic Pathway for Isopropanol Production in Escherichia coli”, Applied and Environmantal Microbiology, 73(24), p. 7814, 2007; T. Jojima, “Production of isopropanol by metabolically engineered Escherichia coli”, Appl. Microbiol. Biotechnol., 77, p. 1219, 2008; US 2009/0246842; PCT 2009/049274; EP2184354). Molar yields of 43.5% are obtained, which is close to the theoretical yield of 50%. One mole of glucose is converted to 2 moles of acetyl-CoA and 2 mole of CO₂. The 2 acetyl-CoA's are condensed to one acetoacetate that is subsequently decarboxylated to make 1 mole of isopropanol and 1 additional CO₂.

Production of acetic acid from glucose by C. thermoaceticum is efficient in that 3 molecules of acetate are produced per molecule of glucose. Two acetate molecules are produced according to the well-known glycolysis, decarboxylation of pyruvate into acetyl-CoA and CO₂, followed by transformation of acetyl-CoA into acetate. During these reactions CO₂ and an excess of electrons are produced. Some microorganisms, such as C. thermoaceticum posses also the Wood-Ljungdahl enzymes that allow converting the CO₂ and excess electrons into more acetate.

$$\frac{\begin{matrix}{C}_{6} {H}_{12} {O}_{6} -> 2 {CH}_{3} COOH + 2 {CO}_{2} + 8 {H}^{+} + \\ 8 {e}^{-} 2 {CO}_{2} + 8 {H}^{+} + 8 {e}^{-} -> {CH}_{3} COOH\end{matrix}}{{C}_{6} {H}_{12} {O}_{6} -> 3 {CH}_{3} COOH}$$

This is particular in that most heterotrophs can only add carbon dioxide to a pre-existing compound and add a carboxyl group. Some bacteria that can form an organic compound directly from carbon dioxide and hydrogen contain hydrogenase, an enzyme that converts hydrogen to two protons and two electrons. These electrons provide the necessary reductive potential for the transformation of carbon dioxide. The overall reaction involves participation of ferredoxin as a reduced electron carrier and the enzymes hydrogenase, carbon monoxide dehydrogenase, and methylenetetrahydrofolate reductase.

### 3. Threonine Degradation

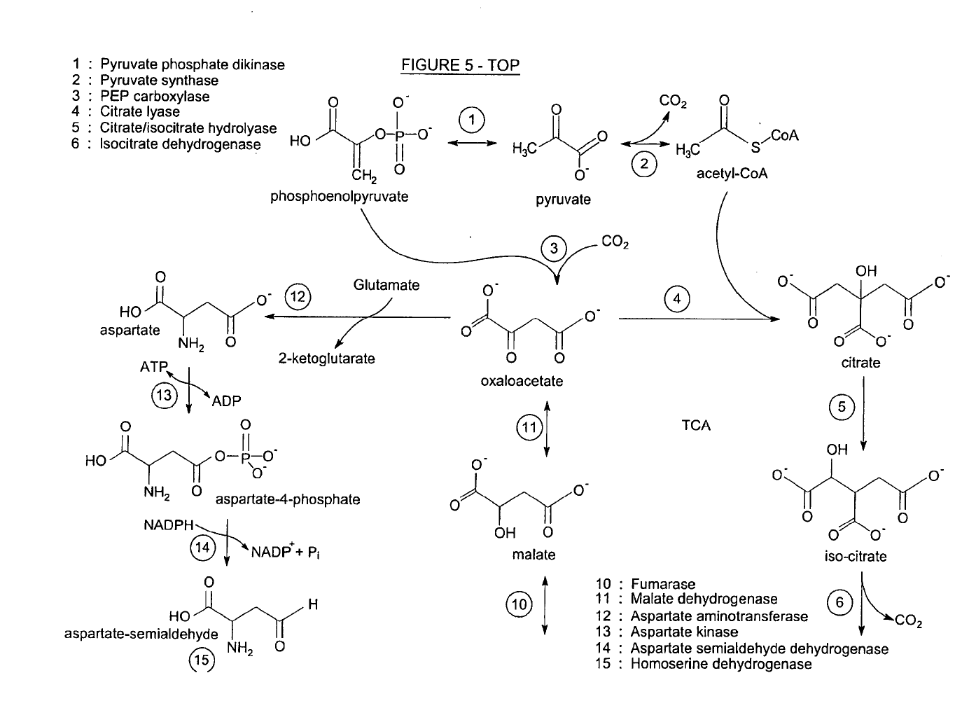

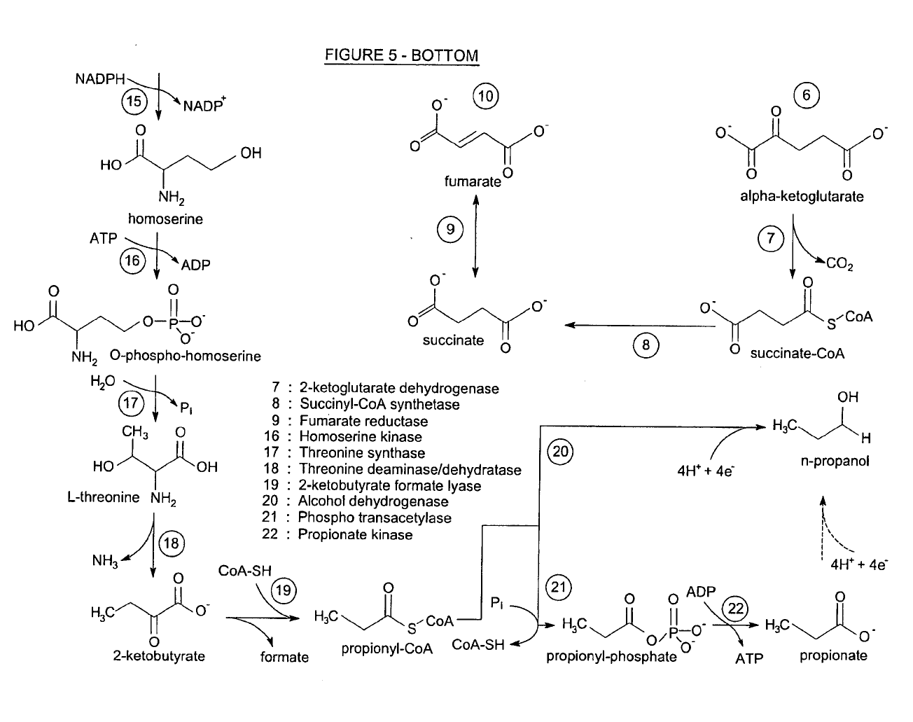

The amino-acids serine and threonine provide the precursors, directly or indirectly, of eight other amino-acids. They also can be derived from one another through the common intermediate glycine. Both serine and threonine can be metabolized in a single enzymic step to energy-rich keto-acids, which can be catabolized to generate ATP by substrate-level phosphorylation, resulting in carboxylic acids. Threonine is derived from oxaloacetate (see FIG. 5) via transamination to make aspartate and serine intermediates. Oxaloacetate is a central metabolite of the TCA cycle that is at the origin of many essential metabolites and can be produced as end-product that condenses again with acetyl-CoA to form citrate. Oxaloacetate needs also to be produced by condensation of PEP with CO₂ with the help of PEP carboxylase, particularly when intermediates of the TCA cycle gets depleted to make other metabolites. One of these derivative metabolites is threonine that is directly produced from oxaloacetate with the help of multiple enzymes (see FIG. 5).

Threonine, a native amino-acid, is known to be fermented by a number of microorganisms like, Clostridium tetanomorphum, Escherichia coli, Salmonella Typhimurium (G. Sawers, “The anaerobic degradation of L-serine and L-threonine in enterobacteria: networks of pathways and regulatory signals”, Arch Microbiol, 171, p. 1-5, 1998; H. Barker, “Amino acid degradation by anaerobic bacteria”, Annual Review of Biochemistry 50, p. 23-40, 1981). The products are typically acetate, propionate, butyrate and 2-aminobutyrate and combinations of the latter. The exploitation of native amino-acid intermediates as final products is often easily implemented in micro-organism as there is no or minor metabolic perturbation because of non toxic intermediates. The basic pathway of threonine fermentation, via 2-ketobutyrate to propionate, is a well-known pathway (G. Gottschalk, “Bacterial Metabolism”, 2^{nd} ed. Springer, 1986). Enteric bacteria possess two types of threonine dehydratases/deaminases—a catabolic enzyme and biosynthetic enzyme that both convert threonine to 2-ketobutyrate (Umbarger H., Brown B., “Threonine deamination in Escherichia coli. II. Evidence for two L-threonine deaminases”, J. Bacteriol., 73(1), p. 105-12, 1957). While the biosynthetic dehydratase is involved in L-isoleucine biosynthesis out of 2-ketobutyrate, the catabolic enzyme participates in the degradation of threonine to propionate, in a pathway that generates ATP, and enables the bacteria to utilize threonine as the sole source of carbon and energy, typically only under anaerobic conditions and when low levels of energy in the cell are available (see FIG. 5). 2-ketobutyrate formate-lyase, is only expressed under anaerobic conditions (Sawers et al.). Once propanoyl-CoA is formed, it is processed via propionyl-P to propionate, in a reaction sequence that produces ATP.

The use of a reductive pathway to make n-propanol to dispose of electrons generated during amino-acid fermentation is unusual. 2-Ketobutyrate is cleaved to yield propionyl-phosphate, CO₂ and H₂ (or formate) by the enzyme, 2-ketobutyrate formate-lyase, in the presence of ferredoxin, CoA-SH, and inorganic phosphate. Clostridium sp. strain 17cr1 is able to ferment L-threonine to propionate and propanol (PH. Janssen, “Propanol as an end product of threonine fermentation”, Arch Microbiol., 182, p. 482-486, 2004). Electrons arising from the oxidation of 2-ketobutyrate to propionyl-CoA are used in reductive pathway leading to propanol formation. When hydrogen is removed from the medium, the formation of propanol ceases.

Also yeasts have the ability to convert amino-acids into the corresponding alcohols (M. Lambrechts, “Yeast and its importance to Wine Aroma—A Review”, S. Afr. J. Enol. Vitic., 21, p. 97, 2000). This occurs according to the Ehrlich pathway where the amino-acids is deaminated to the corresponding 2-ketoacid that is subsequently decarboxylated and further reduced to alcohols. Recently, the 2-ketoacid decarboxylase form Lactococus Lactis and alcohol dehydrogenase from Saccharomyces Cerevisiae have been introduced into E. Coli in order to convert glucose via 2-ketobutyrate into n-propanol (C. Shen, “Metabolic engineering of E. Coli for 1-butanol and 1-propanol production via the keto-acid pathways”, Metabolic Engineering, 10, p. 312, 2008).

### 4. Citramalate Pathway

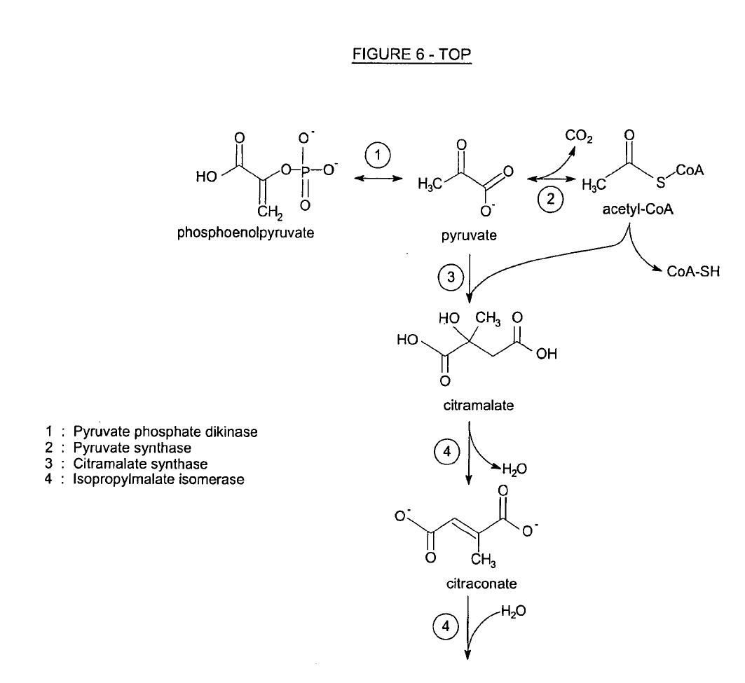

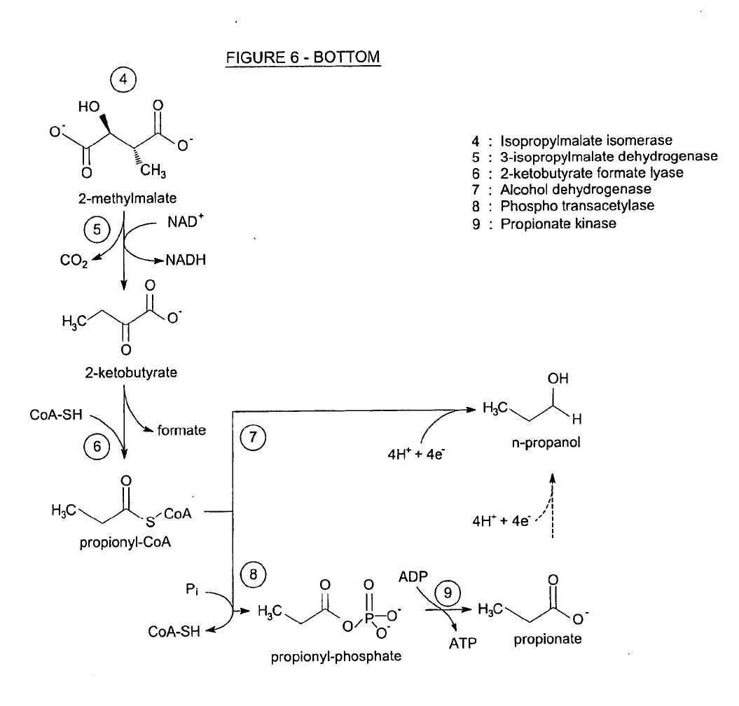

An alternative route to 2-ketobutyrate from pyruvate and acetyl-CoA via citramalate synthase has been reported in several organisms as an alternative pathway for the production of isoleucine, compared to the threonine deamination into 2-ketobutyrate; in archea like Ignicoccus hospitalis and Methanococcus jannaschii, in bacteria like Leptospira interrogans, Geobacter sulfurreducens and in cyanobacteria like Cyanothece sp. ATCC 51142 (B. Wu, “Alternative isoleucine synthesis pathway in cyanobacterial species”, Microbiology, 156, 596-602, 2010; Howell D., “(R)-citramalate synthase in methanogenic archaea.” J. Bacteriol., 181(1), p. 331-3, 1999; J. Huber, “Insights into the autotrophic CO₂ fixation pathway of the archaeon Ignicoccus hospitalis: comprehensive analysis of the central carbon metabolism”, J. Bacteriol., 189(11), p. 4108, 2007). This pathway, designated the citramalate pathway (see FIG. 6) is the most direct route to synthesize 2-ketobutyrate and does not involve transamination followed by deamination. (R)-Citramalate synthesized from pyruvate and acetyl-CoA is then converted to 2-ketobutyrate via an isomerase and dehydrogenase step. As explained for the threonine degradation, the 2-ketobutyrate is the precursor for propionate and propanol. Recently, citramalate synthase from Methanococcus jannaschii has been introduced in Escherichia coli and improved by directed evolution to allow the direct production of n-propanol from glucose via the citramalate pathway (S. Atsumi, “Directed Evolution of Methanococcus jannaschii Citramalate Synthase for Biosynthesis of 1-Propanol and 1-Butanol by Escherichia coli”, Applied and Environmental Microbiology, 74(24), p. 7802-7808, 2008).

### 5. Propanediol Reduction Pathway

1,2-propanediol can be produced by a wide variety of bacteria like, Clostridium thermobutyricum, Escherichia coli, Bacteroides ruminicola and yeasts. Three possible biosynthesis routes are known: (i) in which deoxy-sugars are converted in dihydroxyaceton-phosphate and lactaldehyde that is further reduced to propanediol, (ii) in which the glycolysis intermediate, dihydroxyaceton-phosphate, of regular sugars is converted into methylglyoxal and further reduced to propanediol (G. Bennet, “Microbial formation, biotechnological production and applications of 1,2-propanediol”, Appl. Microbiol. Biotechnol., 55, p. 1, 2001; R. K. Saxena, “Microbial production and applications of 1,2-propanediol”, Indian Journal of Microbiology, 50(1), p. 2-11, 2010).

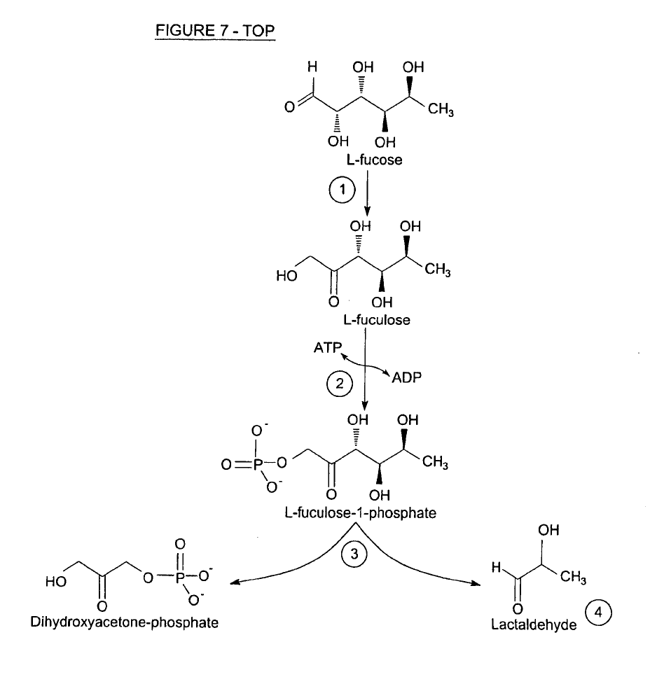

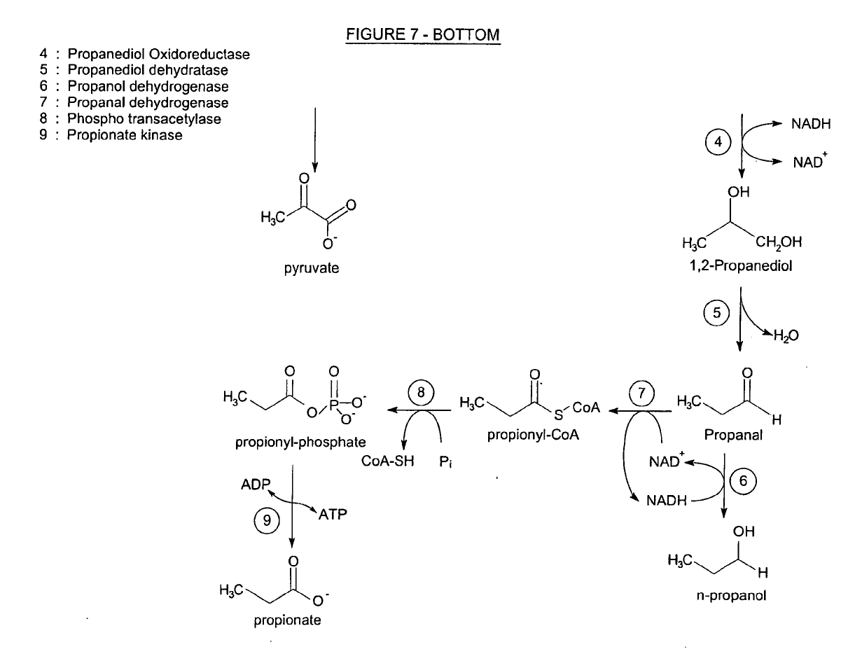

1,2-Propanediol is a fermentation end product of the 6-deoxyhexose sugars L-rhamnose and L-fucose, which are abundantly present in hemicellulose of plant cell walls (Badia, J., “Fermentation mechanisms of fucose and rhamnose in Salmonella typhimurium and Klebsiella pneumonia”, J. Bacteriol., 161, p. 435-437, 1985; Forsberg, “Metabolism of rhamnose and other sugars by strains of Clostridium acetobutylicium and other clostridium species”, Can. J. Microbiol., 33, p. 21, 1987; Tran-Din K., “Formation of 1,2-propanediol and lactate from glucose by Clostridium sperioides under phosphate limitation”, Arch. Microbiol., 142, p. 87, 1985). These methylpentoses are metabolized by pathways that involve an isomerase, a kinase and an aldolase and form equimolar amounts of dihydroxyacetone phosphate (DHAP) and lactaldehyde (see FIG. 7 for fucose fermentation; similar pathway exist for rhamnose). DHAP is converted to pyruvic acid and incorporated into central carbon metabolism. But the fate of the lactaldehyde can differ according to the species and conditions. In presence of oxygen, lactaldehyde is converted to lactate. Under anaerobic conditions, lactaldehyde is reduced to 1,2-propanediol by an NAD-dependent propanediol oxidoreductase.

Several bacteria, including Salmonella, Klebsiella, Shigella, Yersinia, Listeria, Lactobacillus and Lactococcus, include species that grow on 1,2-propanediol in a coenzyme B12-dependent way (E. Sampson, “Microcompartments for B12-Dependent 1,2-Propanediol Degradation Provide Protection from DNA and Cellular Damage by a Reactive Metabolic Intermediate”, J. of Bacteriology, 190(8), p. 2966-2971, 2008). The initial step is carried out by coenzyme B12-dependent propanediol dehydratase, which dehydrates 1,2-propanediol to propionaldehyde. Propionaldehyde is then disproportionated to n-propanol and propionyl-CoA. Propionyl-CoA can be utilized in two different ways depending on the availability of oxygen. Under anaerobic conditions, propionyl-CoA is converted to propionyl phosphate by phosphotransacylase and the propionyl-phosphate is cleaved by propionyl kinase, producing ATP. Both n-propanol and propionate are typically excreted, hence 1,2-propanediol fermentation provides an electron sink and a source for the production of ATP and eventually a three-carbon intermediate (propionyl-CoA) for growth by degradation to pyruvate and succinate via the methylcitrate pathway.

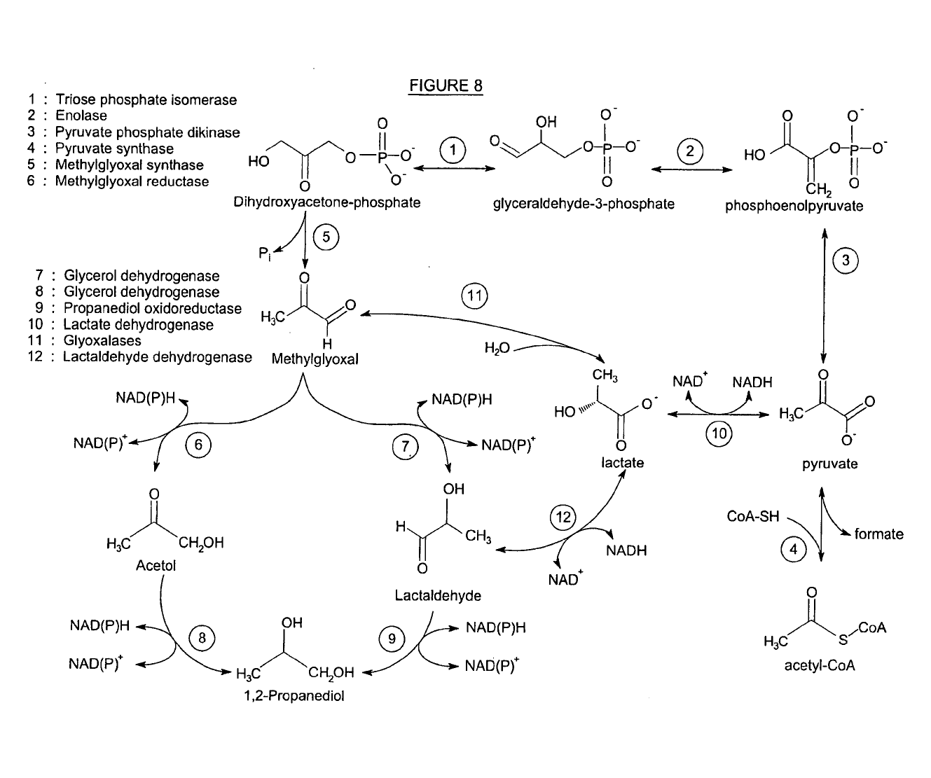

The second route is the metabolism of common sugars (e.g. glucose or xylose) through the glycolysis pathway followed by the methylglyoxal pathway. Dihydroxyacetone phosphate is converted to methylglyoxal that can be reduced either to lactaldehyde or to acetol. These latter compounds can further be reduced yielding 1,2-propanediol (see FIG. 8, many of the involved enzymes have not yet been characterized). This route is used by wild strain producers of 1,2-propanediol, such as Clostridium sphenoides (Tran Din K. and Gottschalk G., Arch. Microbiol. 142: 87-92, 1985) and Thermoanaerobacter thermosaccharolyticum (Cameron D. and Cooney C., Bio/Technology, 4, p. 651-654, 1986; Sanchez-Rivera F, Cameron D., Cooney C., Biotechnol. Lett. 9, p. 449-454, 1987). U.S. Pat. No. 6,303,352 and US 2010/0261239 disclose metabolic engineered microorganism that convert sugars into 1,2 propanediol via the methylglyoxal pathway. There are two possible more direct links between de methylglyoxal pathway and pyruvate via the formation of lactate (see FIG. 8): (i) via glyoxalases enzymes methylglyoxal (which is often toxic for microorganisms) is transformed into lactate and (ii) via lactaldehyde dehydrogenase, lactaldehyde is transformed into lactate (R. Saxena, “Microbial production and application of 1,2-propanediol”, Ind. J. Microbiol., 50(1), p. 2, 2010; J. Weber, “Metabolic flux analysis of Escherichia coli in glucose-limited continuous culture. II. Dynamic response to famine and feast, activation of the methylglyoxal pathway and oscillatory behavior”, Microbiology, 151, p. 707-716, 2005).

The prior art for the production of propanol that can be dehydrated into propylene, described here above, has several technical disadvantages that refrain commercialization, in particular when compared to the ethanol fermentation of sugars, resulting in a theoretical 66.7% carbon efficiency and nearly 100% hydrogen efficiency into useful ethanol (C₆H₁₂O₆→2C₂H₆O+2CO₂):
a. Some of the described pathways involve a decarboxylation step that dispose electron as hydrogen (or formate). This is the case for the aceton/isopropanol pathway where acetoacetate is decarboxylated and the threonine and citramalate pathway where 2-ketobutyrate is decarboxylated. Starting from sugars as feedstock, this reduces the potential yield down to 50%.
b. Some of the described pathways have the potential of a high carbon yield (Wood-Werkman, acrylate pathway, propanediol degradation and threonine degradation), but the pathway is mainly able to provide propionic acid as end product as there is a shortage of reducing equivalents. Often such pathways entail formation of byproducts as acetic acid that consumes less reducing equivalents and hence leave more reducing equivalents to make some propanol.
c. As for the described metabolic pathways the starting feedstock is always a carbohydrate, the potential of biomass valorization is limited to the carbohydrate part of biomass. On top of that the pathways are most of the time not able to use C₅ sugars as starting feedstock whereas biomass contains significant amounts of hemicelluloses, consisting of mainly C₅-sugars.

It is the object of the present invention to provide a process for the production of propylene by gasifying carbonaceous feedstock or reforming natural gas into synthesis gas, converting the synthesis gas into propanol (either n-propanol or isopropanol) by means of a microorganism, possessing the required Wood-Ijundahl enzymes and the enzymes of at least one of the pathways for the production of C₃-oxygenates and dehydrating the propanol into propylene and water. Advantageously it is dehydrated over an acidic catalyst.

Synthesis gas is a mixture of hydrogen, carbonmonoxide and carbondioxide. As the energy carriers are hydrogen and carbonmonoxide, it is recommended that the synthesis gas contains at least hydrogen or carbonmonoxide next to carbondioxide.

By transforming the carbonaceous feedstock into synthesis gas, most of the carbon in the feedstock can be valorized, including the lignin and C₅-sugar constituents of biomass, but also waste carbonaceous feedstock like waste plastics, tires and municipal waste, coal, petcoke, liquid residu's from petrochemical processes like crude refining or liquid residu's from the paper industry and natural gas, associated gas or unconventional gas can be transformed into synthesis gas.

In the above described metabolic pathways, the PEP-pyruvate-acetyl-CoA interconversion (involving the enzymes pyruvate phosphate dikinase/pyruvate kinase and pyruvate dehydrogenase/pyruvate synthase) is the central starting node of the biosynthesis of C₃-oxygenates. Instead of producing these central node intermediates directly from carbohydrates, it is advantageous to produce acetyl-CoA, as part of this node, substantially from synthesis gas via the Wood-Ljungdahl pathway (the reductive acetyl-CoA pathway). Once sufficient acetyl-CoA is produced in the cells, via the central node, PEP-pyruvate-acetyl-CoA, one of the possible pathways described above can result in C₃-oxygenates formation. For biosynthesis pathways that involve decarboxylation to make CO₂ and deposal of electron by hydrogen production or involve decarboxylation to make formate, can be improved with respect to carbon yield as the CO₂/H₂ or formate can be recycled by the Wood-Ljungdahl pathway enzymes.

Via the Wood-Ljungdahl pathway, hydrogen can also provide required electrons via hydrogenase activity for the further reduction of propionic acid, propanediol, propanal or aceton.

It is the object of the present invention to improve the carbon yield for propanol production from carbonaceous feedstock's by optimizing the flow of available electron to the production of propanol by combining the described pathways for making propionic acid, propanediol, propanal, aceton or propanol with the Wood-Ljungdahl pathway (the reductive acetyl-CoA pathway) so that disposed electrons can be recycled via the Wood-Ljungdahl pathway enzymes.

The prior art provides a first indication that Clostridium strain P11 possesses the Wood-Ljungdahl pathway enzyme, to provide the formation of acetyl-CoA and probably the enzymes of the aceton/isopropanol pathway. This strain converts synthesis gas in isopropanol (9.25 g/l) beside ethanol (25.26 g/l), acetic acid (4.82 g/l) and 1-butanol (0.47 g/l) (D. Kundiyana, J. of Bioscience and Bioengineering, 109(5), p. 492, 2010). It has also been reported by E. Caldwell that Clostrium CAT11 and CP19 are able to produce acetate, ethanol, propionate, propanol and butyrate/butanol as end products of fermentation of synthesis gas (M. Caldwell, T. Allen, P. Lawson and R. Tanner, Annual Meeting of the Missouri Valley Branch of the American Society of Microbiology, Mar. 27-28, 2009, University of Kansas, Lawrence, Kans.).

It is the object of the present invention to isolate from nature, wild strain microorganisms that posses the nucleic acid sequence information, to express the enzymes of the Wood-Ljungdahl pathway (reduced Acetyl-CoA pathway) and that posses the nucleic acid sequence information of one of the described pathways to make C₃-oxygenates (aceton/isopropanol pathway, dicarboxylic pathway, acrylate pathway, threonine degradation pathway, citramalate pathway or the propanediol reduction pathway).

It is one object of the present invention to produce propylene by dehydration of propanol that is produced by an improved microorganism that optimizes the carbon flux toward to production of propanol.

U.S. Pat. No. 4,727,214 describes a process for converting anhydrous or aqueous ethanol into ethylene by means of a catalyst of the crystalline zeolite type, said catalyst having, on the one hand, channels or pores formed by cycles or rings of oxygen atoms having 8 and/or 10 elements or members, and on the other hand, an atomic Si/AI ratio of less than about 20. In the examples, the atomic ratio Si/AI of the FER used is from 5 to 20, the temperature from 217 to 280° C., and the WHSV of 2.5 h⁻¹.

JP 2009-215244 A published on 24 Sep. 2009 relates to a method to produce ethylene by contacting ethanol on a H-FER catalyst, having an atomic Si/AI between 3 and 20, more specifically between 4 and 10, sodium and potassium contents both of 0.1% wt or less, more specifically of 0.005% wt or less, the temperature ranging from 200 to 300° C., pressure from 10 to 100 bara, and WHSV from 0.1 to 10 h⁻¹. In examples, the appraisal of the ethanol reaction is achieved by method of gas pulse reaction using gas chromatograph. In the examples the temperature is 260° C. or under.

EP 379803 provides a new process for propylene preparation comprises dehydrating iso-propanol in the presence of a gamma-alumina catalyst having mean pore diameter of 3-15 nm with a standard deviation of 1-4 nm based on a statistical calculation from pore diameter and pore volume Specifically, the gamma-alumina catalyst has pore volume of at least 0.4 cc/g on dry basis; is a low alkali gamma-alumina comprising at least 90 weight % of gamma-alumina, less than 10 weight % of silica and up to 0.5 weight % of alkali metal oxide; and is a weakly acidic gamma-alumina having pKa of 3.3-6.8 measured by Hammett's equation and an integrated acid quantity of up to 0.5 micro-equivalent 1 g on dry basis. The new process gives propylene in high selectivity and yield using a simple non-corrosion resistant reactor at lower temperature than for conventional processes.

WO 2009-098262 (in a first embodiment) relates to a process for the dehydration of an alcohol having at least 2 carbon atoms to make the corresponding olefin, comprising:

introducing in a reactor a stream (A) comprising at least an alcohol, optionally water, optionally an inert component, contacting said stream with a catalyst in said reactor at conditions effective to dehydrate at least a portion of the alcohol to make an olefin,
recovering from said reactor an olefin containing stream (B),

Wherein

the catalyst is:

a crystalline silicate having a ratio Si/AI of at least about 100, or

a dealuminated crystalline silicate, or
a phosphorus modified zeolite,
the WHSV of the alcohols is at least 2 h⁻¹,
the temperature ranges from 280° C. to 500° C.

WO 2009-098262 (in a second embodiment) relates to a process for the dehydration of an alcohol having at least 2 carbon atoms to make the corresponding olefin, comprising:

introducing in a reactor a stream (A) comprising at least an alcohol, optionally water, optionally an inert component, contacting said stream with a catalyst in said reactor at conditions effective to dehydrate at least a portion of the alcohol to make an olefin,
recovering from said reactor an olefin containing stream (B),

Wherein

the catalyst is a phosphorus modified zeolite,
the temperature ranges from 280° C. to 500° C.

### Synthesis Gas Production

Synthesis gas can be produced from low-molecular weight hydrocarbons via reforming or from solid of high-molecular weight liquid hydrocarbons via gasification.

Low-molecular weight hydrocarbons are transformed in presence of steam and/or oxygen into synthesis gas in the “steam reforming” (SMR), CO₂ reforming or autothermal reforming (ATR) (K. Aasberg-Petersen, “Technologies for large-scale gas conversion”, Applied Catalysis A: General, 221, p. 379-387, 2001)

### Steam Reforming

CH₄+H₂O=CO+3H₂

CₙHₘ+nH₂O=nCO+n+(m/2)H₂

CO+H₂O≦CO₂+H₂

### CO₂ Reforming

CH₄+CO₂=2CO+2H₂

### Autothermal Reforming (ATR)

CH₄+1.5O₂=CO+2H₂O

CH₄+H₂O=CO+3H₂

CO+H₂O=CO₂+H₂

Gasification is a partial combustion process that converts carbonaceous materials into CO, CO₂ and H₂. In the gasification reaction, lower than stoichiometric amounts of oxygen (in the form of air, pure oxygen or steam) are fed to the reactor at high temperatures (greater than 700° C.) and therefore the products are only partially reduced (C. Higman and M. van der Burgt, “Gasification”, Elsevier, 2003; Bridgwater A. “Renewable fuels and chemicals by thermal processing of biomass”, Chemical Engineering Journal, 91, p. 87-102, 2003).

In a gasifier, the carbonaceous material is subjected to 4 processes:

(i) Initial heating to dry out any moisture embedded in the carbonaceous solid
(ii) The pyrolysis (or devolatilization) process occurs as the carbonaceous particle heats up. Volatiles are released and char is produced, resulting in significant weight loss. The process is dependent on the properties of the carbonaceous material and determines the structure and composition of the char, which will then undergo gasification reactions.
(iii) The combustion process occurs as the volatile products and some of the char react with O₂ to form CO₂ and CO, which provides heat for the subsequent gasification reactions. Pyrolysis and combustion are very rapid processes.
(iv) The gasification process occurs as the char reacts with CO₂ and steam to produce CO and H₂. The resulting gas is called producer gas (H₂, CO and considerable amounts of CH₄) or syngas (mainly H₂ and CO and little CH₄, so “cleaner”).

The chemistry of coal gasification is quite complex:

Gasification with Oxygen:

C+0.5O₂→CO

C+O₂→CO₂

Gasification with Carbondioxide:

C+CO₂→2CO

Gasification with Steam:

C+H₂O→CO+H₂

Gasification with Hydrogen:

C+2H₂→CH₄

### Water-Gas Shift:

CO+H₂O←→H₂+CO₂

### Methanation:

CO+2H₂→CH₄+H₂O

Gasification consists of a series of controlled chemical reactions taking place at up to 70 bar or more and temperature as high as 1400° C. As the feedstock is exposed to rising temperature in the gasifier, devolatilization and breaking of weaker chemical bonds occur, yielding tars, oils, phenols, and hydrocarbon gases. These products generally react further to form CO, H₂ and lesser quantities of CO₂. The fixed carbon that remains after devolatilization is gasified through reactions with O₂, water, CO₂ and H₂ and these gases react further to produce the final gas mixture. The water-gas shift reaction alters the H₂ to CO ratio of the final gas mixture. Methanation reactions are favored by high pressures and low temperatures. Since no O₂ is consumed, exothermic (heat releasing) methanation reactions increase the efficiency of gasification and the heating value of the syngas (producer gas) produced but reduced the hydrogen to carbonmonoxide ratio which is preferentially as high as possible for sequence chemical synthesis.

Under the substoichiometric reducing conditions of gasification, most of the feedstock sulfur converts to hydrogen sulfide (H₂S) and some converts to carbonyl sulfide (COS). Nitrogen chemically bound in the feed generally converts to gaseous nitrogen (N₂), with some ammonia (NH₃) and a small amount of hydrogen cyanide (HCN) also being formed. Chlorine in the feed is primarily converted to hydrogen chloride (HCl), with some appearing as chloride-containing particulates. Trace elements associated with both organic and inorganic components of coal feedstock, such as mercury (Hg) and arsenic (As), appear in the various ash fractions as well as in gaseous emissions and need to be removed from the syngas prior to further use.

### BRIEF SUMMARY OF THE INVENTION

In one embodiment of the present invention is provided:

A process for making propylene by dehydration of propanol, involving the following steps:
1. Gasifying carbonaceous solid or liquid feedstock or reforming gaseous carbonaceous feedstock into synthesis gas,
2. Removing contaminants from the synthesis gas
3. Fermenting the synthesis gas by means of a microorganism into substantially propanol in which the microorganism
   1. Is a wild strain having the natural capability to ferment synthesis gas into substantially propanol or
   2. Is a microorganism, possessing the required nucleic acid sequence information to express the enzymes for the biosynthesis of C₃-oxygenates (aceton/isopropanol pathway, dicarboxylic pathway, acrylate pathway, threonine degradation pathway, citramalate pathway or the propanediol reduction pathway), modified by conferring it with the required nucleic acid sequence information to express the enzymes of the Wood-Ljungdahl pathway (reduced Acetyl-CoA pathway), or
   3. Is a microorganism, possessing the required nucleic acid sequence information to express the enzymes of the Wood-Ljungdahl pathway (reduced Acetyl-CoA pathway), modified by conferring it with the required nucleic acid sequence information to express the enzymes for the biosynthesis of C₃-oxygenates (aceton/isopropanol pathway, dicarboxylic pathway, acrylate pathway, threonine degradation pathway, citramalate pathway or the propanediol reduction pathway).
4. Fractionating and purifying the stream containing predominantly propanol,
5. Dehydrating said above stream in a reactor at conditions effective to dehydrate at least a portion of the propanol to make propylene,
6. recovering from said reactor the propylene containing stream

It is another embodiment of the present invention to provide

A process for making propylene by dehydration of propanol, involving the following steps:
1. Gasifying carbonaceous solid or liquid feedstock or reforming gaseous carbonaceous feedstock into synthesis gas,
2. Removing contaminants from the synthesis gas
3. Co-Fermenting the synthesis gas with at least one liquid oxygenate, like carbohydrates, glycerol, propanediol, lactaldehyde, lactic acid, acetol, methanol, ethanol, acetic acid, acetaldehyde, propionic acid, propanal and aceton, by means of a microorganism into substantially propanol in which the microorganism
   1. Is a wild strain having the natural capability to ferment synthesis gas into substantially propanol or
   2. Is a microorganism, possessing the required nucleic acid sequence information to express the enzymes for the biosynthesis of C₃-oxygenates (aceton/isopropanol pathway, dicarboxylic pathway, acrylate pathway, threonine degradation pathway, citramalate pathway or the propanediol reduction pathway), modified by conferring it with the required nucleic acid sequence information to express the enzymes of the Wood-Ljungdahl pathway (reduced Acetyl-CoA pathway), or
   3. Is a microorganism, possessing the required nucleic acid sequence information to express the enzymes of the Wood-Ljungdahl pathway (reduced Acetyl-CoA pathway), modified by conferring it with the required nucleic acid sequence information to express the enzymes for the biosynthesis of C₃-oxygenates (aceton/isopropanol pathway, dicarboxylic pathway, acrylate pathway, threonine degradation pathway, citramalate pathway or the propanediol reduction pathway).
4. Fractionating and purifying the stream containing predominantly propanol,
5. Dehydrating said above stream in a reactor at conditions effective to dehydrate at least a portion of the propanol to make propylene,
6. recovering from said reactor the propylene containing stream

The stream containing predominantly propanol can contain other alcohols e.g. ethanol, butanols.

The fermentation or the co-fermentation step (3), produces a mixture of alcohols, containing at least 50 wt % of propanol, between 0 and 50 wt % of ethanol and between 0 and 50 wt % of butanol.

In an embodiment the weight ratio of said alcohols to the propanol is less than 20/80.

In an embodiment the weight ratio of said alcohols to the propanol is less than 10/90. In an embodiment the weight ratio of said alcohols to the propanol is less than 5/95.

In an embodiment dehydration is made over an acidic catalyst at a temperature of at least 200° C. and a WHSV of at least 1 h⁻¹. Dehydration can be made by introducing in a reactor a stream comprising at least propanol, optionally water, optionally an inert component, contacting said stream with a catalyst in said reactor at conditions effective to dehydrate at least a portion of the propanol to make propylene.

By dehydration is made an olefin having the same number of carbons as the alcohol to be dehydrated. Of course if other alcohols are present the dehydration produces the corresponding olefin (ethanol leads to ethylene and butanol leads to butene).

### DETAILED DESCRIPTION OF THE INVENTION

As regards the production of synthesis gas, it can be produced from low-molecular hydrocarbons by reforming or from high-molecular weight liquid hydrocarbons or solid carbonaceous solid via gasification.

Essentially, synthesis gas is formed via steam reforming reaction and partial oxidation of natural gas (Dybkjaer I., “Tubular reforming and autothermal reforming of natural gas—an overview of available processes”, Fuel. Proc. Tech., 42, p. 85, 1995; Moulijn J., “Chemical Process Technology”, Wiley & Sons, 2001). In steam reformer (SMR) a catalyst is packed in tubes placed in a fired heater. The reformer tubes are heated externally by burners using typically natural gas. The feedstock to the reformer is a mixture of desulfurised natural gas and steam (>3:1 steam to carbon ratio in order to prevent coking of the catalyst). Steam methane reforming is endothermic and the molar ratio of hydrogen to carbon monoxide produced by SMR is approximately 3:1. If CO₂ (CO₂ reforming or dry-reforming) is added to the feedstock the H₂/CO ratio can be reduced. When the feedstock contains heavy hydrocarbons, a prereformer is used to first reform the heavy hydrocarbons.

The autothermal reformer (ATR) reactor has a compact design consisting of a burner, combustion chamber and catalyst bed placed in a refractory lined pressure vessel as. The hydrocarbon feedstock is reacted with a mixture of oxygen and steam in a sub-stoichiometric flame. The steam to carbon ratio can be as low as 0.6. In the fixed catalyst bed, the synthesis gas is further equilibrated. The composition of the product gas will be determined by the thermodynamic equilibrium at the exit pressure and temperature, which is determined through the adiabatic heat balance based on the composition and flows of the feed, steam and oxygen added to the reactor. The produced synthesis gas is completely soot-free.

In the combined reformer (or 2-step reformer), the tubular reformer (SMR) is combined with a secondary reformer, acting as an ATR, to which oxygen is added.

There are four main types of gasifiers currently used commercially: counter-current fixed bed, co-current fixed bed, fluid bed and entrained flow (C. Higman and M. van der Burgt, “Gasification”, Elsevier, 2003).

### Counter-Current Fixed Bed (Updraft, Also Called Moving Bed Gasifiers):

A fixed bed of carbonaceous feedstock (coal, petcoke, char or biomass) entering at the top with a counter current flow of steam, oxygen and/or air flows up through the solid bed. The feedstock is dried by the syngas leaving the chamber while the gas leaving the gasifier is cooled by the entering feedstock. In case of oxygen-containing feedstock like biomass, char that is generated continues moving down the gasifier vessel where it is reduced and starts reacting with the oxygen and CO₂. The dried feedstock travels down the vessel where gasification of coal or char occurs. The ash is then removed dry or as slag. Gas exit temperatures are low, which is good for thermal efficiency, but this increases the tar and methane impurities in the synthesis gas (McKendry P., “Energy production from biomass (part 1): overview of biomass”, Bioresource Technology 83, 37-46, 2002; Bridgwater T., “Review Biomass for energy”, Journal of the Science of Food and Agriculture 86, p. 1755-1768, 2006). The advantages of updraft gasifiers are their simple construction, low cost, ability to handle high moisture and inorganic content, and their high energy efficiency due to the lower temperature of the gas leaving the chamber.

### Co-Current Fixed Bed (Downdraft):

This is similar to the counter-current gasifier described above beside that the steam, oxygen and/or air flows co-currently down with the solid carbonaceous feedstock bed. Some heat has to added to the top of the gasifier by combustion or heat exchange. Because the gas passes through the hot zone combusting the tars and leaving the reactor from the bottom hence the final product has a higher purity. The exit temperature of the gas is higher, resulting in a lower overall efficiency. It has a fairly simple design and is low cost, and it produces a relatively cleaner gas with very low tar make. Some of the disadvantages are that the system requires low moisture and ash feedstock and it has low efficiency because the product gas leaves the gasifier at higher temperatures, which requires an additional cooling system as compared to an updraft gasifier.

### Fluid Bed:

The carbonaceous feedstock is fluidized in oxygen/air and steam. The feedstock size is reduced to a small particle size, and is mixed eventually with the fluidizing material, which is usually silica sand, ceramic, or alumina. The oxidizer and the solid crushed feedstock enter the reactor from the bottom, where a hot bed forms, where most of the conversion to synthesis gas occurs. The ash is removed as a dry product as it becomes defluidized. Fuel throughput is higher than for a fixed bed, and has the advantage of uniform temperature distribution achieved in the gasification zone resulting in cleaner reactions. However, overall conversion can be very high, the gas stream entrains a lot of fine particulates which has be separated from the gas and recycled back to the gasifier (circulating fluidized bed). Fluidized beds work particularly well for biomass, as it has a lot of highly corrosive ash that would harm the fixed bed reactors.

Entrained flow: a dry pulvurized solid, an atomized liquid fuel or a fuel slurry is gasified with O₂ (or air) in co-current flow. The gasification reactions take place in a dense cloud of very fine particles. Most coals are suitable for this type of gasifier because of the high operating temperatures (well above ash melting point, to assure high carbon conversion) and because the coal particles are well separated from one another. The high temperatures and pressures also mean that a higher throughput can be achieved (short contact time), however thermal efficiency is somewhat lower as the gas must be cooled before it can be cleaned with existing technology. The high temperatures also mean that tar and CH₄ are not present in the product gas; however the O₂ requirement is higher than for the other types of gasifiers. All entrained flow gasifiers remove the major part of the ash as a slag as the operating temperature is well above the ash fusion temperature. Some fuels (in particular certain types of biomass) can form slag that is corrosive for the ceramic inner walls that serve to protect the gasifier outer wall. However some entrained bed type of gasifiers do not have a ceramic inner wall but have an inner water or steam cooled wall covered with partially solidified slag and hence do not suffer from corrosive slags.
As regards the microorganism, they can include prokaryotic and eukaryotic microbial species from the Domains Archaea, Bacteria and Eucarya (including yeast and filamentous fungi, protozoa, algae, or higher Protista). The term “prokaryotes” refers to cells which contain no nucleus or other cell organelles and are generally classified in one of two domains, the Bacteria and the Archaea. The ultimate difference between organisms of the Archaea and Bacteria domains is based on fundamental differences in the nucleotide base sequence in the 16S ribosomal RNA.

The microorganisms of the present invention are either naturally occurring microorganisms or metabolically engineered microorganism.

Naturally occurring microorganism of the present invention are isolated from nature, so called wild strains that have the ability to ferment synthesis gas into propanol. Such wild strains can be improved by random screening and rationalized selection. Random mutagenesis is based on repeated applications of three steps: (i) mutagenesis with chemicals or radiation of a population to induce genetic variability, (ii) screening from the surviving population to identify an improved strain under small-scale standard fermentation testing and (iii) assay of fermentation test for confirmation of improved strains (S. Parekh, “Improvement of microbial strains and fermentation processes”, Appl. Microbiol. Biotechnol., 54, p. 287, 2000). Furthermore, adaptive evolution can be applied to make the microorganism more resistance to certain external conditions, like higher solvent tolerance, higher fermentation temperature, higher syngas pressure etc.

Random mutagenesis may be performed using a suitable physical or chemical mutagenising agent. Examples of a physical or chemical mutagenesing agent suitable for the present purpose include, but are not limited to, UV irradiation, ionizing irradiation such as gamma irradiation, hydroxylamine, N-methyl-N′-nitro-N-nitrosoguanidine (MNNG), O-methyl hydroxylamine, nitrous acid, ethyl methane sulphonate (EMS), sodium bisulfite, and nucleotide analogues. When such agents are used the mutagenesis is typically performed by incubating the cell to be mutagenised in the presence of the mutagenising agent of choice under suitable conditions, and selecting for cells showing a significantly increased or decreased production of the targeted molecule(s).

The term “metabolically engineered” entails rational designed pathway and assembly of biosynthetic genes, genes associated with operons, and control elements of such nucleic acid sequences, for the production of a desired metabolite, in particular as end-product propanol (isopropanol or n-propanol). It further embraces optimization of metabolic flux by regulation and optimization of transcription, translation, protein stability and protein functionality, functional in an optimal manner under the applied fermentation conditions. The biosynthetic genes can be heterologous to the host microorganism, either by virtue of being foreign to the host, or being modified by mutagenesis, recombination, and/or association with a heterologous expression control sequence in an endogenous host cell. Accordingly, “metabolically engineered” microorganisms are created via the introduction of genetic material into a host microorganism of choice thereby acquiring new properties, e.g. the ability to produce a new or greater quantity of metabolite. It also encompasses the activation of endogenous nucleic acid sequences encoding a target enzyme through genetic modification of e.g., a promoter sequence in a host microorganism and the introduction of exogenous nucleic acid sequences encoding a target enzyme into a host microorganism. The introduction of genetic material (containing gene(s), or parts of genes, coding for one or more of the enzymes involved in a biosynthetic pathway for the production of isopropanol or n-propanol and eventually including additional elements for the expression (or regulation) of these genes, e.g. promoter sequences) into a host microorganism results in a new or improved ability to produce isopropanol or n-propanol from synthesis gas. A “metabolite” refers to any substance produced by anabolic or catabolic metabolism. An important intermediate metabolite is Acetyl-CoA, produced from synthesis gas or other oxygenated compounds like carbohydrates, ethanol, acetic acid, required for the synthesis of the desired end-metabolite isopropanol or n-propanol.

In order to express the metabolic pathway of interest and/or reorienting metabolic flux, the host cell of the invention could be genetically modified by using standard technologies known to the person skilled in the art. For example, if we want to delete a pathway to increase metabolic flux for the production of propanol, the gene sequences responsible for production of enzymes of endogenous pathway may be inactivated or partially or entirely eliminated. The inactivation could be obtained by modification of the respective structural or regulatory regions (such as genes). Known and useful techniques include, but are not limited to, specific or random mutagenesis, PCR generated mutagenesis, site specific DNA deletion, insertion and/or substitution, gene disruption or gene replacement, anti-sense techniques, or a combination thereof.

The term “heterologous” or “exogenous” indicates enzymes or nucleic acids that are expressed in an organism other than the organism from which they originated. The term “endogenous” indicates enzymes and nucleic acids that are expressed in the organism in which they originated.

The resulting “recombinant microorganism” is a host microorganism that has been genetically modified to express or over-express endogenous nucleic acid sequences, or to express non-endogenous nucleic acid sequences, such as those included in a vector (encoding a target enzyme or enzymes involved in a metabolic pathway for producing a desired metabolite). It is understood that the terms “recombinant microorganism” refer also to the progeny.

The term “host microorganism” represents (i) a cell that occurs in nature, i.e. a “wild-type” cell that has not been genetically modified or (ii) a cell that has been genetically modified but which does not express or over-express a target enzyme (such microorganism can act as a host in the generation of a microorganism modified to express or over-express another target enzyme). Methods of over-expressing in microorganisms are well known in the art, and any such method is contemplated for use in the construction of the microorganisms of the present invention.

Any method can be used to introduce an exogenous nucleic acid molecule into microorganisms and many such methods are well known to those skilled in the art: for example, transformation, electroporation, conjugation, and fusion of protoplasts are common methods for introducing nucleic acid into microorganisms (J. Dale, “Molecular Genetics of bacteria”, 4^{th} ed., Wiley, 2004; C. Smolke, “The Metabolic Pathway Engineering Handbook”, CRC Press, 2010).

The exogenous nucleic acid molecule transposed to a host microorganism can be maintained within that cell in any form: for example, exogenous nucleic acid molecules can be incorporated into the genome of the cell or maintained in an episomal state (extra-chromosomal genetic elements, like plasmids) that can be passed stably on to daughter cells. The microorganisms described herein can contain a single copy, or multiple copies of a particular exogenous nucleic acid molecule.

Methods for expressing enzymes from an exogenous nucleic acid molecule are well known to those skilled in the art, including, without limitation, assembling a nucleic acid sequence such that a regulatory element promotes the expression of a nucleic acid sequence that encodes the desired enzyme. Typically, regulatory elements (promoters, enhancers, etc) are nucleic acid sequences that regulate the expression of other nucleic acid sequences at the level of transcription.

Additionally, when expression of certain enzymatic activity is to be repressed or eliminated, the gene for the relevant enzyme, protein or RNA can be eliminated by known deletion techniques. These deletion techniques are useful to further improve the yield of isopropanol or n-propanol by eliminated the pathways leading to byproducts, like C₂ or C₄ oxygenates or hydrogen. Heterologous control elements can be used to repress expression of endogenous genes. Deletion techniques can be used for optimized both the wild strain and the metabolically engineered microorganisms.

It is an embodiment of the present invention, that synthesis gas can be co-fermented with oxygenated compounds, like carbohydrates, glycerol, propanediol, lactaldehyde, lactic acid, acetol, methanol, ethanol, acetic acid, acetaldehyde, propionic acid, propanal and aceton. The carbohydrates are essentially “biomass derived sugars” and includes, but is not limited to, molecules such as glucose, mannose, fucose, rhamnose, xylose, and arabinose, lactose, sorbose, fructose, idose, galactose, mannose and dimers, oligomers or polymers of the latter (starch, cellulose, hemicelluloses and pectins).

As regards the fermentation, this is known per se and documented in literature (P. Munasinghe, “Biomass-derived syngas fermentation into biofuels: Opportunities and challenges”, Bioresource Technology, 101(13), p. 5013, 2010; Chapter 11 & 13 of “Bioenergy and Biofuel from biowastes and biomass”, S. Khanal ed., ASCE, 2010; J. Williams, “Keys to bioreactor selection”, Chemical Engineering Progress, p. 34, March 2002). Syngas fermentation can be carried out in both batch and continuous-flow bioreactors. In batch reactors, the gaseous substrate is introduced in closed system bioreactor where the syngas is supplied continuously but the products remain in the reactor or can be withdrawn at a selected time during fermentation. Bubble column reactors, monolithic biofilm reactors, membrane bioreactors and trickling bed reactors are some of the other common bioreactors. In some of the bioreactors microbubble spargers can be implemented which enhances the mass transfer in two ways (M. Bredwell, P. Srivastava and R. M. Worden, “Reactor design issues for synthesis-gas fermentations”, Biotechnology Progress, 15, p. 834-844, 1999). Firstly, decreasing bubble sizes cause internal pressure increases, leading to an increase in the driving force. Secondly, the steady state liquid phase concentration gradient at the surface of the bubble is inversely proportional to the diameter. Many other essential techniques (fermentation kinetics, preservation of microorganism, nutrients media, sterilization, inocula development and fermentor design) to implement fermentation are described in reference books (P. Stanbury, “Principles of fermentation technology”, Elsevier Science, 2003; B. McNeil, “Practical Fermentation Technology”, John Wiley & Son, 2008)
a. The continuous stirred-tank reactor is the most common bioreactor employed in syngas fermentation. A CSTR has a continuous flow of gas through a constant liquid volume. The liquid consists of a suspension of the microorganism and a dilute solution of essential nutrients for the microorganism to grow and survive and a make-up of such liquid nutrient solution and/or microorganism suspension is continuously or intermittently added. Syngas is bubbled through a sparger and an agitation is applied for enhanced mass transfer between the two phases. The fermentation product is withdrawn from the system at the same flow rate as the feed entering the reactor. Cell-recycle systems can be used in conjunction with the CSTR to increase cell-density within the reactor. In such a system, the fermentation broth is pumped through a recycle filter, decanter or centrifuge and the retentate (containing the microorganism cells) is separated from the permeate (cell-free media) and recycled to the bioreactor. An enhanced degree of agitation or mixing is accomplished by baffled impellers to enhance the mass transfer between the substrate and the microorganisms. Higher rotational speeds of the impellers tend to break the gas bubbles into finer ones thereby increasing the interfacial area between the syngas and the aqueous suspension of the microorganisms.
b. Bubble column reactors are most suitable for very large volume industrial applications. These reactors have a large height-to-diameter ratio (aspect ratio) in which high mass transfer can be obtained even without the use of additional agitation. Smaller bubble size and improved gas dispersion can be obtained by using special devices to disperse the syngas in the bottom of the bioreactor, like porous fritted discs or multiple sparger rings to disperse the syngas. Gas-lift reactors are like bubble columns containing a draught tube. Gas is dispersed into the bottom of a central draught tube that controls the circulation of gas and the culture medium. Gas rises through the tube, forming bubbles, entraining the liquid suspension and exhaust gas disengages at the top of the column. The degassed liquid then flows downward external to the draught tube and the product is drained from the tank. As there is an induced controlled flow direction of the liquid suspension, the tube can be designed to serve as an internal heat exchanger, or a heat exchanger can be added to an internal circulation loop. A variation on this gaslift reactor is the external gaslift reactor consisting of an internal riser section and an external downcomer. Through the riser the gas rises and induces the liquid suspension upwards. At the top, the gas disengages and the liquid suspension returns via the external downcomer back to the bottom of the reactor. The downcomer can serve as a heat exchanger if required.
c. Monolithic biofilm reactors, is like a bundle of small channels made out of solid porous material on which a biofilm of microorganisms grows. It exhibits a good mass-transfer of gas to the biofilm and a low pressure drop. During the operation, attached microorganisms in the biofilm utilize the gaseous substrates to produce fermentation products. Monolith reactors have high mass-transfer characteristics in particular in multiphase operation. The liquid (typically aqueous solution containing the nutrients and fermentation products) moves as a thin film over the channel surface and the gas flow through the core of the channels. When liquid flow rate are high or gas flow rate low, the gas and liquid move through the channel as separate slugs (Taylor flow). The gas bubble nearly completely fills the diameter of the channel, leaving only a very thin film of liquid near the stagnant biofilm, consequently a high gas-stagnant biofilm mass-transfer is possible. The appropriate liquid/gas ratio in order to reach Taylor flow can be installed by recirculating the liquid around the monotlih.
d. Trickle-bed reactors are packed beds, continuous reactor in which the liquid culture flows down, trickling over the surface of the packing, while maximizing the gas-liquid interface. The syngas is allowed to move either downward (co-current) or upward (counter-current) direction. Since these types of reactors do not require mechanical agitation, the power consumption of trickle-bed reactors is lower than the CSTR.
e. Membrane-based bioreactor consists of membranes (flat sheet (FS), multitubular or hollow fiber (HFM)) that allow effectively facilitating the mass transfer in aqueous culture media (K. Lee and B. Rittmann, “Applying a novel autohydrogenotrophic hollow-fiber membrane biofilm reactor for denitrification of drinking water”, Water Research, 36, p. 2040-2052, 2001; S. Judd, “The MBR Book”, Elsevier, 2006; US patent 2009/0215163). In the membrane reactor, syngas is diffused through the walls of membranes without forming bubbles. The microbial community grows and settles as a biofilm on the wall of the membranes where it continuously ferments H₂ and CO to alcohols. These membrane bioreactors can be operated under high pressure with higher mass transfer rates and reduced reactor volumes.

As regards the propanol fractionation and purification, the propanol as the main product of the fermentation is fractionated by any conventional means, like stripping (flashing) of the dissolved gases in the fermentation broth, like filtration, decantation and/or centrifugation to remove solids (microorganism and any other precipitated material) from the aqueous solution, like distillation (stripping and/or rectification) in order to concentrate the propanol stream or like membrane separation in order to isolate the propanol from the aqueous solution or fermentation broth.

Before going to the dehydration reactor, the propanol (and eventually ethanol and butanol) can be purified by the process of WO 2010/060981 the content of which is incorporated in the present application. It describes a process for the purification of an alcohol in the course of a process comprising: (1) providing a reaction zone (C) comprising an acid type catalyst; (2) providing a reaction zone (B) comprising an acid adsorbent material; (3) providing an alcohol stream comprising impurities; (4) introducing the alcohol stream of (3) into the reaction zone (B) and bringing said stream into contact with the acid adsorbent material at conditions effective to reduce the amount of impurities having an adverse effect on the acid type catalyst of the reaction zone (C); (5) recovering from step (4) an alcohol stream and introducing it into the reaction zone (C); (6) optionally introducing one or more reactants (R) into the reaction zone (C); (7) operating said reaction zone (C) at conditions effective to recover a valuable effluent.

As regards the propanol dehydration, dehydration of alcohols is known per se. Alcohols dehydration has been described in WO-2009-098262 and WO-2009-098268 the content of which are incorporated in the present application.

WO-2009-098262 relates to a process for the dehydration of at least an alcohol to make at least an olefin, wherein the catalyst is:
a crystalline silicate having a ratio Si/AI of at least about 100, or
a dealuminated crystalline silicate, or
a phosphorus modified zeolite, the WHSV of the alcohols is at least 2 h⁻¹, the temperature ranges from 280° C. to 500° C. It relates also to the same process as above but wherein the catalyst is a phosphorus modified zeolite and at any WHSV.

WO-2009-098268 relates to a process for the dehydration of at least an alcohol to make at least an olefin, comprising:
a) introducing in a reactor a stream (A) comprising at least an alcohol optionally in aqueous solution and an inert component,
b) contacting said stream with a catalyst in said reactor at conditions effective to dehydrate at least a portion of the alcohol to make an olefin,
c) recovering from said reactor a stream (B) comprising: the inert component and at least an olefin, water and optionally unconverted alcohol,
d) optionally fractionating the stream (B) to recover the unconverted alcohol and recycling said unconverted alcohol to the reactor of step a),
e) optionally fractionating the stream (B) to recover the inert component and the olefin and recycling said inert component to the reactor of step a), wherein, the inert component is selected among ethane, the hydrocarbons having from 3 to 10 carbon atoms, naphtenes and CO₂, the proportion of the inert component is such as the reactor operates essentially adiabatically. It also relates to a similar process as above but the catalyst is:
   a crystalline silicate having a ratio Si/AI of at least 100, or
   a dealuminated crystalline silicate, or
   a phosphorus modified zeolite, the WHSV of the alcohol is at least 2 h⁻¹ wherein the catalyst is a crystalline silicate having a ratio Si/AI of at least 100 or a dealuminated crystalline silicate. Advantageously the pressure of the dehydration reactor is high enough to help the recovery of the inert component and recycling thereof in the reactor of step a) without a gas compressor but only a pump.

As regards the propanol stream, the propanol may be subjected to dehydration alone or in mixture with an inert medium. The inert component is any component provided there is no adverse effect on the catalyst. Because the dehydration is endothermic the inert component can be used to bring energy. The inert component may be selected among the saturated hydrocarbons having up to 10 carbon atoms, naphtenes, nitrogen and CO₂. Advantageously it is a saturated hydrocarbon or a mixture of saturated hydrocarbons having from 3 to 7 carbon atoms, more advantageously having from 4 to 6 carbon atoms and is preferably pentane. An example of inert component can be any individual saturated compound, a synthetic mixture of the individual saturated compounds as well as some equilibrated refinery streams like straight naphtha, butanes etc. Advantageously the inert component is a saturated hydrocarbon having from 3 to 6 carbon atoms and is preferably pentane. The weight proportions of respectively propanol, water and inert component are, for example, 5-100/0-95/0-95 (the total being 100). The alcohol stream, containing substantially propanol may also contain ethanol and butanols. The propanol concentration within the alcohols should be at least 50 wt %; the ethanol concentration between 0 and 50 wt % and the butanol between 0 and 50 wt %.

As regards the dehydration reactor, it can be a fixed bed reactor, a moving bed reactor or a fluidized bed reactor. A typical fluid bed reactor is one of the FCC type used for fluidized-bed catalytic cracking in the oil refinery. A typical moving bed reactor is of the continuous catalytic reforming type. The dehydration may be performed continuously in a fixed bed reactor configuration using a pair of parallel “swing” reactors. The various preferred catalysts of the present invention have been found to exhibit high stability. This enables the dehydration process to be performed continuously in two parallel “swing” reactors wherein when one reactor is operating, the other reactor is undergoing catalyst regeneration. The catalyst of the present invention also can be regenerated several times.

As regards the dehydration pressure, it can be any pressure but it is more easy and economical to operate at moderate pressure. By way of example the pressure of the reactor ranges from 0.5 to 30 bars absolute (50 kPa to 3 MPa), advantageously from 0.5 to 5 bars absolute (50 kPa to 0.5 MPa), more advantageously from 1.2 to 5 bars absolute (0.12 MPa to 0.5 MPa) and preferably from 1.2 to 4 bars absolute (0.12 MPa to 0.4 MPa). Advantageously the partial pressure of the alcohols (propanol and eventually ethanol and butanol) is from 1.2 to 4 bars absolute (0.12 MPa to 0.4 MPa), more advantageously from 1.2 to 3.5 bars absolute (0.35 MPa).

As regards the dehydration temperature, and the first embodiment it ranges from 200° C. to 600° C., advantageously from 300° C. to 580° C., more advantageously from 350° C. to 580° C. As regards the temperature and the second embodiment it ranges from 320° C. to 600° C., advantageously from 320° C. to 580° C., more advantageously from 350° C. to 580° C.

These reaction temperatures refer substantially to average catalyst bed temperature. The propanol dehydration is an endothermic reaction and requires the input of reaction heat in order to maintain catalyst activity sufficiently high and shift the thermodynamic equilibrium to sufficiently high conversion levels.

In case of fluidised bed reactors: (i) for stationary fluidised beds without catalyst circulation, the reaction temperature is substantially homogeneous throughout the catalyst bed; (ii) in case of circulating fluidised beds where catalyst circulates between a converting reaction section and a catalyst regeneration section, depending on the degree of catalyst backmixing the temperature in the catalyst bed approaches homogeneous conditions (a lot of backmixing) or approaches plug flow conditions (nearly no backmixing) and hence a decreasing temperature profile will install as the conversion proceeds.

In case of fixed bed or moving bed reactors, a decreasing temperature profile will install as the conversion of the alcohols proceeds. In order to compensate for temperature drop and consequently decreasing catalyst activity or approach to thermodynamic equilibrium, reaction heat can be introduced by using several catalyst beds in series with interheating of the reactor effluent from the first bed to higher temperatures and introducing the heated effluent in a second catalyst bed, etc. When fixed bed reactors are used, a multi-tubular reactor can be used where the catalyst is loaded in small-diameter tubes that are installed in a reactor shell. At the shell side, a heating medium is introduced that provides the required reaction heat by heat-transfer through the wall of the reactor tubes to the catalyst.

As regards the dehydration WHSV of the alcohols, it ranges advantageously from 1 to 20 h⁻¹, preferably from 3 to 15 h⁻¹, more preferably from 4 to 10 h⁻¹.

As regards the dehydration effluent stream, it comprises essentially water, olefin, the inert component (if any) and unconverted alcohols. Said unconverted alcohols is supposed to be as less as possible. The olefin is recovered by usual fractionation means. Advantageously the inert component, if any, is recycled in the stream (A) as well as the unconverted alcohols, if any. Unconverted alcohols, if any, is recycled to the reactor in the stream (A).

As regards the dehydration catalyst, it is by way of example, a crystalline silicate of the group FER (ferrierite, FU-9, ZSM-35), MWW (MCM-22, PSH-3, ITQ-1, MCM-49), EUO (ZSM-50, EU-1), MFS (ZSM-57), ZSM-48, MTT (ZSM-23), MFI (ZSM-5 or silicalite), MEL (ZSM-11) or TON (ZSM-22, Theta-1, NU-10),

or a dealuminated crystalline silicate of the group FER (ferrierite, FU-9, ZSM-35), MWW (MCM-22, PSH-3, ITQ-1, MCM-49), EUO(ZSM-50, EU-1), MFS (ZSM-57), ZSM-48, MTT (ZSM-23), MFI (ZSM-5 or silicalite), MEL (ZSM-11) or TON (ZSM-22, Theta-1, NU-10),
or a phosphorus modified crystalline silicate of the group FER (ferrierite, FU-9, ZSM-35), MWW (MCM-22, PSH-3, ITQ-1, MCM-49), EUO (ZSM-50, EU-1), MFS (ZSM-57), ZSM-48, MTT (ZSM-23), MFI (ZSM-5 or silicalite), MEL (ZSM-11) or TON (ZSM-22, Theta-1, NU-10),
or a silicoaluminophosphate molecular sieve of the group AEI, CHA or AEL,
or gamma-, beta-, eta- or delta-alumina and amorphous alumina
or silica-alumina
or a alkalized, silicated, zirconated or titanated or fluorinated alumina.

The crystalline silicate can be subjected to various treatments before use in the dehydration including, ion exchange, modification with metals (in a not restrictive manner alkali, alkali-earth, transition, or rare earth elements), external surface passivation, modification with phosphorus-compounds, steaming, acid treatment or other dealumination methods, or combination thereof.

Another suitable catalyst for the present process is the silicoaluminophosphate molecular sieves of the AEI, CHA or AEL group with typical example the SAPO-18, SAPO-34 or SAPO-11 molecular sieve. The SAPO molecular sieve is based on the ALPO, having essentially an Al/P ratio of 1 atom/atom. During the synthesis silicon precursor is added and insertion of silicon in the ALPO framework results in an acid site at the surface of the micropores of the 10-membered ring sieve. The silicon content ranges from 0.1 to 10 atom % (Al+P+Si is 100).

In another specific embodiment the crystalline silicate or silicoaluminophosphate molecular sieve is mixed with a binder, preferably an inorganic binder, and shaped to a desired shape, e.g. pellets. The binder is selected so as to be resistant to the temperature and other conditions employed in the dehydration process of the invention. The binder is an inorganic material selected from clays, silica, metal silicates, metal oxides such as ZrO₂ and/or metals, or gels including mixtures of silica and metal oxides. If the binder which is used in conjunction with the crystalline silicate is itself catalytically active, this may alter the conversion and/or the selectivity of the catalyst. Inactive materials for the binder may suitably serve as diluents to control the amount of conversion so that products can be obtained economically and orderly without employing other means for controlling the reaction rate. It is desirable to provide a catalyst having a good crush strength. This is because in commercial use, it is desirable to prevent the catalyst from breaking down into powder-like materials. Such clay or oxide binders have been employed normally only for the purpose of improving the crush strength of the catalyst. A particularly preferred binder for the catalyst of the present invention comprises silica. The relative proportions of the finely divided crystalline silicate material and the inorganic oxide matrix of the binder can vary widely. Typically, the binder content ranges from 5 to 95% by weight, more typically from 20 to 75% by weight, based on the weight of the composite catalyst. Such a mixture of the crystalline silicate and an inorganic oxide binder is referred to as a formulated crystalline silicate. In mixing the catalyst with a binder, the catalyst may be formulated into pellets, extruded into other shapes, or formed into spheres or a spray-dried powder. Typically, the binder and the crystalline silicate are mixed together by a mixing process. In such a process, the binder, for example silica, in the form of a gel is mixed with the crystalline silicate material and the resultant mixture is extruded into the desired shape, for example cylindrical or multi-lobe bars. Spherical shapes can be made in rotating granulators or by oil-drop technique. Small spheres can further be made by spray-drying a catalyst-binder suspension. Thereafter, the formulated crystalline silicate is calcined in air or an inert gas, typically at a temperature of from 200 to 900° C. for a period of from 1 to 48 hours.

Another family of suitable catalysts for the dehydration of propanol into propylene are alumina's (amorphous, gamma-, beta-, eta- or delta-alumina's) as such or alumina's that have been modified by surface treatment with alkali's, silicon, zirconium or titanium and silica-alumina's. Alumina's are generally characterised by a rather broad acid strength distribution and having both Lewis-type and Bronsted-type acid sites. The presence of a broad acid strength distribution makes the catalysis of several reactions, requiring each a different acid strength, possible. This often results in low selectivity for the desired product. Deposition of alkali's, silicon, zirconium or titanium on the surface of alumina allows rendering the catalyst significantly more selective. For the preparation of the alumina based catalyst, suitable commercial alumina's can be used, preferably eta or gamma alumina, having a surface area of 10 to 500 m²/gram. The catalyst according to the present invention is prepared by adding 0.05 to 10% of alkali (earth) metal, silicon, zirconium or titanium. The addition of these metals can be done during the preparation of the alumina or can be added to the existing alumina, eventually already activated. Addition of the metal during the preparation of the alumina can be done by dissolving the metal precursor together with the aluminium precursor before precipitation of the final alumina or by addition of the metal precursor to the aluminium hydroxide gel. A preferred method is adding metal precursors to the shaped alumina. Metal precursors are dissolved in a suitable solvent, either aqueous or organic, and contacted with the alumina by incipient wetness impregnation or by wet impregnation or by contacting with an excess of solute during a given time, followed by removing the excess solute. The alumina can also be contacted with vapour of the metal precursor. Suitable metal precursors are halides of alkali (earth) metals, silicon, zirconium or titanium, oxyhalides of zirconium or titanium; alcoxides of alkali (earth) metals, silicon, zirconium or titanium; acetates, oxalates or citrates of alkali (earth) metals, zirconium or titanium; nitrates, sulfates or phosphates of alkali (earth) metals, or mixtures of the above. The solvent is selected according to the solubility of the metal precursor. The contacting can be done at temperature of 0° C. to 500° C., most preferred from 10° C. to 200° C. After the contacting, the alumina is eventually washed, dried and finally calcined in other to enhance the surface reaction between the alkali (earth) metals, silicon, zirconium or titanium and the alumina and the removal of the metal precursor ligands. The use of alkalized, silicated, zirconated or titanated or fluorinated alumina's for the dehydration of propanol into propylene is preferably done in the presence of water. The weight ratio of water to propanol ranges from 1/25 to 3/1. Fluorinated alumina is known in itself and can be made according to the prior art.

One skilled in the art will also appreciate that the propylene made by the dehydration process of the present invention can be, by way of example, polymerized into polypropylene, copolymers of ethylene, hexane, octane or decene and propylene, ethylene-propylene rubbers (EPR), ethylene-propylene-diene polymers (EPDM), can be used for alkylation of benzene to make cumene, a precursor for phenol production, can be oxidized to acrylic acid, can be ammoxidised into acrylonitrile, can be oxidized to propylene oxide for making polyether polyols and propanediol and can be hydroformylated to n-butyraldehyde.

The present invention relates also to said polypropylene, ethylene-propylene rubbers (EPR), ethylene-propylene-diene polymers (EPDM), cumene, acrylic acid, acrylonitrile, propyleneoxide and n-butyraldehyde.

1. A process for making propylene by dehydration of propanol, comprising:
1. gasifying a carbonaceous solid or liquid feedstock or reforming gaseous carbonaceous feedstock into synthesis gas,
2. removing contaminants from the synthesis gas,
3. fermenting the synthesis gas by means of a microorganism into a stream comprising substantially propanol in which the microorganism
i. is a wild strain having the natural capability to ferment synthesis gas into substantially propanol or
ii. is a microorganism possessing the required nucleic acid sequence information to express the enzymes for the biosynthesis of C₃-oxygenates, modified by conferring it with the required nucleic acid sequence information to express the enzymes of the Wood-Ljungdahl pathway, or
iii. is a microorganism possessing the required nucleic acid sequence information to express the enzymes of the Wood-Ljungdahl pathway, modified by conferring it with the required nucleic acid sequence information to express the enzymes for the biosynthesis of C₃-oxygenates,
4. fractionating and purifying the stream containing predominantly propanol,
5. dehydrating said stream in a reactor at conditions effective to dehydrate at least a portion of the propanol to make propylene,
6. recovering from said reactor a stream containing the propylene.

2. A process for making propylene by dehydration of propanol, comprising:
1. gasifying a carbonaceous solid or liquid feedstock or reforming a gaseous carbonaceous feedstock into synthesis gas,
2. removing contaminants from the synthesis gas,
3. co-fermenting the synthesis gas with at least one liquid oxygenate by means of a microorganism into a stream comprising substantially propanol in which the microorganism
i. is a wild strain having the natural capability to ferment synthesis gas into substantially propanol or
ii. is a microorganism, possessing the required nucleic acid sequence information to express the enzymes for the biosynthesis of C₃-oxygenates, modified by conferring it with the required nucleic acid sequence information to express the enzymes of the Wood-Ljungdahl pathway or
iii. is a microorganism possessing the required nucleic acid sequence information to express the enzymes of the Wood-Ljungdahl pathway, modified by conferring it with the required nucleic acid sequence information to express the enzymes for the biosynthesis of C₃-oxygenates,
4. fractionating and purifying the stream containing predominantly propanol,
5. dehydrating said stream in a reactor at conditions effective to dehydrate at least a portion of the propanol to make propylene,
6. recovering from said reactor a stream containing the propylene.

3. A process according to claim 1, wherein the fermentation step (3) produces a mixture of alcohols, containing at least 50 wt % of propanol, between 0 and 50 wt % of ethanol and between 0 and 50 wt % of butanol and where in the fractionation and purification step (4) the propanol, ethanol and butanol are fractionationed and purified and where in the dehydration step (5) propanol in presence of ethanol and butanol, if any, are dehydrated simultaneously and where in the recovery step (6) the propylene and ethylene and butenes, if any, are recovered from said reactor.

4. A process according to claim 1, wherein the C₃-oxygenates are produced via a combination of the Wood-Ljungdahl pathway and the aceton/isopropanol pathway, involving at least an acetyl-CoA synthase, a pyruvate synthase and acetoacetate decarboxylase, and producing mainly isopropanol.

5. A process according to claim 1, wherein the C₃-oxygenates are produced via a combination of the Wood-Ljungdahl pathway and the dicarboxylic pathway, involving at least an acetyl-CoA synthase, a pyruvate synthase and an methylmalonyl-CoA carboxytransferase or an propionyl-CoA succinate transferase, and producing mainly n-propanol.

6. A process according to claim 1, wherein the C₃-oxygenates are produced via a combination of the Wood-Ljungdahl pathway and the acrylate pathway, involving at least an acetyl-CoA synthase, a pyruvate synthase and an acryloyl-CoA reductase and producing mainly n-propanol.

7. A process according to claim 1, wherein the C₃-oxygenates are produced via a combination of the Wood-Ljungdahl pathway and the threonine degradation pathway, involving at least an acetyl-CoA synthase, a pyruvate synthase and a 2-ketobutyrate formate lyase and producing mainly n-propanol.

8. A process according to claim 1, wherein the C₃-oxygenates are produced via a combination of the Wood-Ljungdahl pathway and the citramalate pathway, involving at least an acetyl-CoA synthase, a pyruvate synthase and a citramalate synthase and producing mainly n-propanol.

9. A process according to claim 1, wherein the C₃-oxygenates are produced via a combination of the Wood-Ljungdahl pathway and the propanediol reduction pathway, involving at least an acetyl-CoA synthase, a pyruvate synthase, a propanediol oxidoreductase and a propanediol dehydratase and producing mainly n-propanol.

10. A process according to claim 1, wherein the dehydration is made over an acidic catalyst at a temperature of at least 200° C. and a WHSV of at least 1 h⁻¹.

11. A process according to claim 10 wherein dehydration is be made by introducing in the reactor a stream comprising at least the propanol, optionally water, optionally an inert component, contacting said stream with a catalyst in said reactor at conditions effective to dehydrate at least a portion of the propanol to make the propylene.

12. Use of the propylene made according to claim 1.

13. Use of the propylene made according to claim 1, wherein the propylene is a copolymer containing ethylene, hexene, octene or decene as a comonomer.

14. Use of the propylene made according to claim 1 to make ethylene-propylene rubbers (EPR).

15. Use of the propylene made according to claim 1 to make ethylene-propylene-diene copolymers (EPDM).

16. Use of the propylene made according to claim 1 to make acrylonitrile.

17. Use of the propylene made according to claim 1 to make acrylic acid.

18. Use of the propylene made according to claim 1 to make cumene.

19. Use of the propylene made according to claim 1 to make propylene-oxide.

20. Use of the propylene made according to claim 1 to make n-butyraldehyde.
